(19) United States
(12) Patent Application Publication
(10) Pub. No.: US 2026/0261623 A1
(43) Pub. Date: Sep. 3, 2026

(54) AUTOMATED CHROMOGENIC IN SITU HYBRIDIZATION QUANTIFICATION AND IMMUNOHISTOCHEMICAL ASSESSMENT FROM WHOLE SLIDE IMAGES

(71) Applicants: Memorial Sloan-Kettering Cancer Center, New York, NY (US); Memorial Hospital for Cancer and Allied Diseases, New York, NY (US); Sloan-Kettering Institute For Cancer Research, New York, NY (US)

(72) Inventors: Yukako Yagi, New York, NY (US); Dara S. Ross, New York, NY (US); Chie Ohnishi, New York, NY (US); Takashi Ohnishi, New York, NY (US)

(21) Appl. No.: 18/838,097

(22) PCT Filed: Feb. 13, 2023
(86) PCT No.: PCT/US2023/012954

§ 371 Date: Aug. 13, 2024

Related U.S. Application Data
(60) Provisional application No. 63/309,933, filed on Feb. 14, 2022.

(51) Int. Cl. H04N 1/60; G02B 21/34; G02B 21/36; G06V 20/69; G16H 30/40

(52) U.S. Cl. CPC H04N 1/6044; G02B 21/34; G02B 21/365; G06V 20/695; G16H 30/40

(57) ABSTRACT

Systems and methods for performing color calibration using control tissue to automate detection are disclosed. The system can receive calibration images from a scanner. The calibration images can each depict a portion of a stained tissue sample having a known score value. The system can determine a correction function for the scanner based on the plurality of calibration images. The system can receive an unscored slide image depicting a portion of stained tissue. The unscored slide image may be captured by the scanner. The system can present, in a graphical user interface of an application executed by the system, a score for the unscored slide image generated using the correction function for the scanner.

### CROSS-REFERENCE TO RELATED APPLICATIONS

This application claims the benefit of and priority to U.S. Provisional Patent Application No. 63/309,933, filed Feb. 14, 2022, the content of which is incorporated by reference herein in its entirety for all purposes.

### TECHNICAL FIELD

The present application relates generally to color calibration across slide images to reduce inaccuracy in determinations of cellular or tissue abnormalities.

### BACKGROUND

Differences in colors detected in stains may be used to detect the presence of abnormal tissue. However, slide-imaging quality may vary by machine, location, and scanning technology. Therefore, a slide scanned at a first scanning location at a first time may appear to indicate color differences to another scan of the same slide at a second scanning location, or at the same scanning location at a future time. These inconsistencies present challenges when attempting to automatically detect tissue or cellular irregularities, such as markers for cancer cells.

### SUMMARY

The systems and methods of the present disclosure provide techniques for calibrating the color differences in a slide across different scanning devices and across different time periods through the use of a control tissue and/or calibration slides. The control tissue or calibration slides can be scanned at a certain time, and a color mapping, which maps different colors to corresponding DAB (3,3′-diaminobenzidine) concentrations, can be generated. The color map can be stored in association with a timestamp for the day that the tissue was scanned and the scanned slide image of the control tissue or the calibration slides. The color map (e.g., provided on a calibration slide) can then be used to calibrate other scans of other tissue samples, to automate detection of cellular features of interest. By utilizing the present techniques, color differences across institutions or scanners no longer impact the accuracy of automatic detection. Therefore, the systems and methods described herein provide improvements to scanning technology.

At least one aspect of the present disclosure relates to a method for performing color calibration using control tissue or calibration slides to automate detection. The method can be performed, for example, by one or more processors coupled to a non-transitory memory. The method includes receiving a plurality of calibration images from a scanner, each of the plurality of calibration images depicting a portion of a respective stained tissue sample having a known score value. The method includes determining, by the one or more processors, a correction function for based on the plurality of calibration images. The method includes receiving an unscored slide image depicting a portion of stained tissue, the unscored slide image captured by the scanner. The method includes presenting, in a graphical user interface of an application executing on the one or more processors, a score for the unscored slide image generated using the correction function period.

In some implementations, the method includes determining a score for the unscored slide image based on the intensity profile. In some implementations, each of the plurality of calibration images are selected to correspond to a respective score value. In some implementations, determining the intensity profile comprises performing a color calibration process. In some implementations, the color calibration process comprises calculating a color correction matrix that is applied to the unscored slide image. In some implementations, determining the intensity profile comprises determining one or more color thresholds corresponding to the plurality of calibration images.

In some implementations, the method includes annotating, by the one or more processors, the unscored slide image based on the intensity profile. In some implementations, determining the intensity profile comprises determining one or more intensity values over a multiple day period. In some implementations, the method includes receiving a second unscored slide image generated by a second scanner during a second time period. In some implementations, the method includes identifying a second intensity profile associated with the second scanner and the second time period. In some implementations, the method includes presenting the second unscored slide image in the application with the second intensity profile. In some implementations, the plurality of calibration images correspond to a respective HER2 score.

At least one other aspect of the present disclosure is directed to a system for performing color calibration using control tissue or calibration slides to automate detection. The system can include one or more processors coupled to a non-transitory memory. The system can receive a plurality of calibration images from a scanner, each of the plurality of calibration images depicting a portion of a respective stained tissue sample having a known score value. The system can determine a correction function for the scanner based on the plurality of calibration images. The system can receive an unscored slide image depicting a portion of stained tissue, the unscored slide image captured by the scanner. The system can present, in a graphical user interface of an application executing on the one or more processors, a score for the unscored slide image generated using the correction function.

In some implementations, the system can determine a score for the unscored slide image based on the correction function. In some implementations, each of the plurality of calibration images are selected to correspond to a respective score value. In some implementations, to determine the correction function, the system can perform a color calibration process. In some implementations, the system can calculate a color correction matrix that is applied to the unscored slide image. In some implementations, to determine the correction function, the system can determine one or more color thresholds corresponding to the plurality of calibration images. In some implementations, the system can annotate the unscored slide image based on the correction function. In some implementations, to determine the correction function, the system can determine one or more intensity values over a multiple day period.

In some implementations, the system can receive a second unscored slide image generated by a second scanner during a second time period. In some implementations, the system can identify a second correction function associated with the second scanner and the second time period. In some implementations, the system can present the second unscored slide image in the application with the second correction function. In some implementations, the plurality of calibration images each correspond to a respective HER2 score.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. It will be readily appreciated that features described in the context of one aspect of the invention can be combined with other aspects. Aspects can be implemented in any convenient form, for example, by appropriate computer programs, which may be carried on appropriate carrier media (computer readable media), which may be tangible carrier media (e.g. disks) or intangible carrier media (e.g. communications signals). Aspects may also be implemented using suitable apparatus, which may take the form of programmable computers running computer programs arranged to implement the aspect.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1 depicts an example system for performing color calibration using control tissue or calibration slides to automate detection, in accordance with one or more implementations;

FIG. 2 depicts an example system architecture diagram showing a process for processing stained slide images using the color calibration techniques described herein, in accordance with one or more implementations;

FIGS. 3A and 3B show a comparison between different scans of the same stained slide performed on different scanners, in accordance with one or more implementations;

FIG. 4 shows a side-by-side comparison of immunohistochemistry (IHC) calibration slide sets used in connection with the present techniques, in accordance with one or more implementations

FIG. 5 shows views of a calibration slide including various stain intensities, in accordance with one or more implementations;

FIG. 6 shows a side-by-side comparison of a color mapping, which maps stain intensities to colors, and corresponding stained slides, in accordance with one or more implementations;

FIG. 7 shows a diagram of estimated 3,3′-Diaminobenzidine (DAB) concentration being used as a threshold of scoring slide images, in accordance with one or more implementations;

FIG. 8 shows a side-by-side comparison of scoring images of stains having varying intensities, in accordance with one or more implementations;

FIG. 9 shows a color bar (e.g., a color mapping) displayed in a user interface with a whole-slide image (WSI) of a tissue sample, in accordance with one or more implementations;

FIG. 10 shows example calibrator results over a five day period, including color mappings between DAB concentrations and color intensities, in accordance with one or more implementations;

FIG. 11 shows example cell pallet results over a five day period, including color mappings between DAB concentrations and color intensities, in accordance with one or more implementations;

FIG. 12 shows an example assignment of an intensity profile (e.g., a color mapping) to one or more slide images, in accordance with one or more implementations;

FIG. 13 shows an example color bar displayed in a user interface with images of WSI having different intensities, in accordance with one or more implementations;

FIG. 14 depicts an example method for performing color calibration using control tissue or calibration slides to automate detection, in accordance with one or more implementations;

FIG. 15 is a block diagram of a server system and a client computer system in accordance with an illustrative embodiment;

FIG. 16 shows views of an example calibration slide including various stain intensities, in accordance with one or more implementations;

FIG. 17 shows views of an example color chart slide including various reference color, in accordance with one or more implementations;

FIGS. 18A and 18B show an example flow diagram of a process for color and intensity standardization, including scanner calibration and staining calibration, which may be implemented by the system of FIG. 1, in accordance with one or more implementations;

FIG. 19 shows an example flow diagram of a process that may be implemented by the system of FIG. 1 for scanner calibration, in accordance with one or more implementations;

FIGS. 20A and 20B show an example flow diagram of a process for staining calibration, which may be implemented by the system of FIG. 1, in accordance with one or more implementations;

FIGS. 21A, 21B, and 21C show example slide images, plots, and mean and variance values, respectively, for example stained breast cancer tissue images, in accordance with one or more implementations;

FIGS. 22A, 22B, and 22C show example slide images, plots, and mean and variance values, respectively, for example HER IHC stained breast cancer tissue images, in accordance with one or more implementations;

FIGS. 23A, 23B, and 23C show example slide images, plots, and mean and variance values, respectively, for example IHC calibrator images, in accordance with one or more implementations;

FIGS. 24A, 24B, and 24C show comparisons of dE* maps with and without brown in HER2 IHC stained breast cancer tissue images, in accordance with one or more implementations;

FIGS. 25A, 25B, and 25C show comparisons of dE* maps with and without brown in IHC stained calibrator images, in accordance with one or more implementations;

FIGS. 26A, 26B, and 26C show comparisons of dE* maps with and without brown in H&E stained breast cancer tissue images, in accordance with one or more implementations;

FIGS. 27A and 27B show HER2 IHC breast cancer images scanned using a 40× objective lens with intensity color bar, in accordance with one or more implementations;

FIG. 28 shows a comparison of tissue images and dE* map, in accordance with one or more implementations;

FIGS. 29A and 29B depict HER2 IHC stained samples using a 40× objective lens, in accordance with one or more implementations;

FIGS. 30A, 30B, 30C and 30D depict HER2 IHC stained breast cancer images scanned at 0.23 μm/pixel resolution (40× equivalent) for a 0 case, a 1+ case, a 2+ case, and a 3+ case, respectively, in accordance with one or more implementations;

FIGS. 31, 31B, and 31C depict an example HER2 IHC stained IHC calibrator, in accordance with one or more implementations;

FIG. 32 depicts a flow diagram of an example process, that may be implemented by the system described in connection with FIG. 1, to perform standardization between slide images, in accordance with one or more implementations;

FIG. 33 depicts a data flow diagram of a process for calculating of the thresholds of stain intensities from tissue images, in accordance with one or more implementations;

FIG. 34 depicts a data flow diagram of a first implementation of the process described in connection with FIG. 32 including correcting reference thresholds to adapt to original tissue images, in accordance with one or more implementations;

FIG. 35 depicts a data flow diagram of a second implementation of the process described in connection with FIG. 32 including correcting the color and intensity of tissue images, in accordance with one or more implementations;

FIGS. 36A, 36B, 36C, and 36D depict examples of color correction in 2+ cases, in accordance with one or more implementations; and

FIGS. 37A, 37B, 37C, and 37D depict examples of color correction in 3+ cases, in accordance with one or more implementations.

### DETAILED DESCRIPTION

The present techniques can allow for performing color calibration using control tissue or calibration slides, which enables for increased accuracy when automating detection across different institutions, or the same institution across varying time periods. It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes. For the purpose of better understanding the present disclosure, a brief overview of the sections of the detailed description may be helpful:

Immunohistochemistry (IHC) staining is used as a process for detecting markers for various cellular abnormalities, including cancer. Generally, IHC analysis measures the intensity of stains in a WSI to detect the presence (or absence) of a target protein. One such protein is the HER2 protein. The IHC test for the HER2 protein measures the amount of HER2 receptor protein on the surface of cells in a breast cancer tissue sample. This measurement is often reflected in the form of a score, which may range from 0 to 3+. If the score is 0 to 1+, the tissue sample may be referred to as “HER2 negative.” If the score is 2+, the tissue sample may be referred to as borderline.” If the score is 3+, the tissue sample is referred to as “HER2 positive.” HER2 negative, borderline, and HER2 positive breast cancer all may have different treatment plans. Therefore, it is important to ensure the accuracy of these scores when determining the best course of action for patient treatment.

In HER2 IHC staining, DAB stains the membrane region brown, and hematoxylin stains the nuclear region blue or purple for tissue counterstaining. During image analysis, a score is classified based on intensity and percentage of membrane staining. HER2 status is visually assessed by pathologists using microscope usually, but tends to be subjective and reproducibility in interpretation has become a critical issue. Modern guidelines recommend image analysis using digital slide (e.g., WSI) to enhance objectivity and accuracy of HER2 IHC assessments.

However, calculating these scores is dependent on the quality of the WSI scan. Various factors may influence the accuracy of the scoring process when analyzing a stained tissue sample. Generally, the HER2 score for a given slide is calculated based on the intensity of the stain present in a stained WSI. The intensity of colors for WSI, however, may vary for a number of reasons. For example, the intensity may vary based on Intensity is varied by institution, the day or time of stain, the particular scanner used to perform the scan, or the scanning protocol used in the scan, among others.

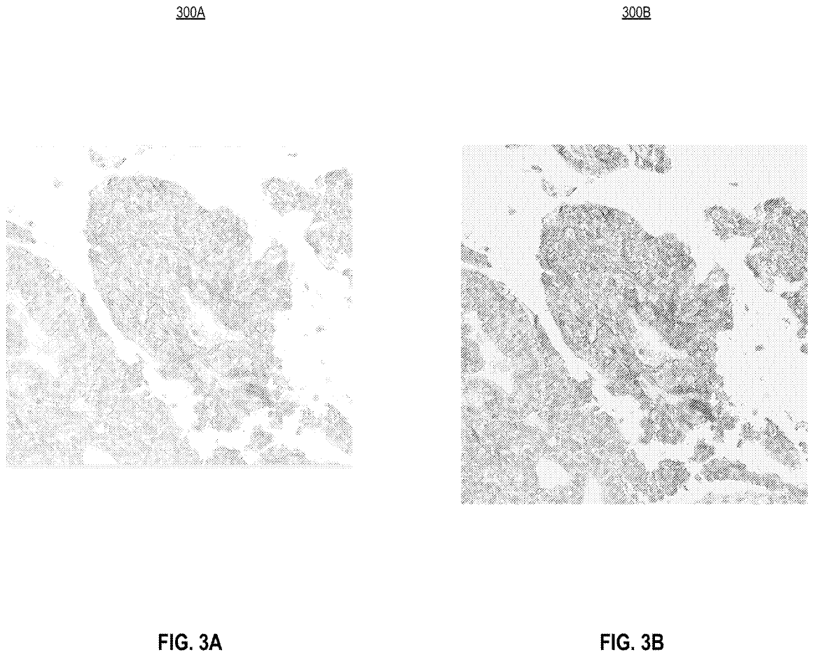

These differences can produce stark differences in scoring. An example of such differences is shown in the side-by-side comparison of the same tissue sample captured using different scanners shown in FIGS. 3A and 3B. As shown in FIG. 3A, the scan appears very light, and would likely be scored in the 0 to 1+ range (e.g., HER2 negative). However, the same tissue sample scanned using a different scanner shown in FIG. 3B is much darker, and would likely result in a score of 2+ or even 3+, indicating a borderline or HER2 positive case. Each of these different outcomes would result in a difference course of treatment for the patient.

Color intensity variation is therefore an important issue in pathology. Variability in histochemical staining is known to affect the accuracy and reproductivity in clinical practice and research. Small differences in staining intensity can significantly affect the interpretation of IHC slides, particularly with membrane stains, resulting in differences in diagnoses and treatment outcomes. Additionally color intensity variation is not confined to irregularities within the staining process. Digitization of the slides using WSI scanners may introduce further variation of color. Such variations affect the image analysis for HER2 test.

It is therefore advantageous for a system to automatically compensate for these differences, such that scans of the same tissue sample across different institutions, time periods, or scanning technology would not affect the HER2 scoring, or other processes that rely on determining an intensity of IHC staining in a WSI. The systems and methods of the present disclosure solve these and other issues by utilizing a control tissue or a calibration slide, as described herein, to perform a color calibration process for tissues scanned on a particular scanner. The control tissue can be a set of calibration slides that are used to calibrate scanning devices for certain staining processes (e.g., a HER2 test, etc.). The calibration slides can be used to create a color mapping for a particular scanner at a particular time period, which maps different stain intensities (e.g., colors) to known HER2 scores (e.g., 0, 1+, 2+, 3+, etc.). This color mapping can then be used to score other stained slides captured on the same scanner around the same time period.

The color mapping can be created by testing the calibration slides over a time period (e.g., five days, one week, etc.). These and other features are described in greater detail herein. The calibration slides may include, for example, peptide-coated microbeads with different concentrations to achieve different colors or shades of colors. The calibration slides may include, for example, peptide-coated microbeads with different concentrations to achieve different colors or shades of colors. The calibration slides can be stained together with tissue sample slides to obtain the color intensity of the stain correlated with those of the test slides, as described herein. The color standardization or normalization methods described herein can be applied to address the issues of color variation. In the color standardization for IHC, the intensity of DAB can be calibrated using the techniques described herein.

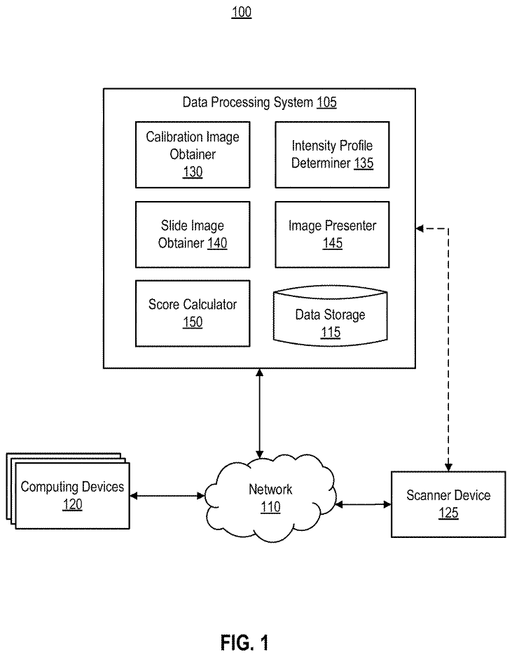

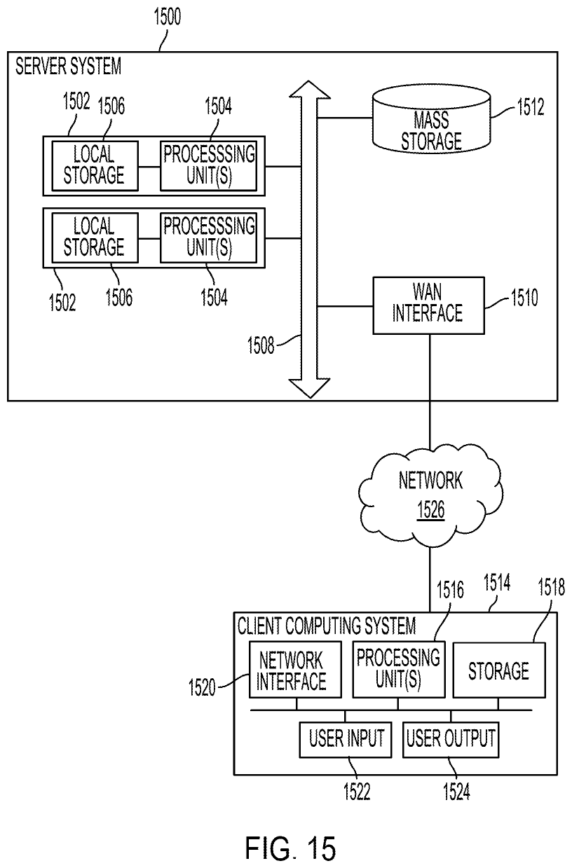

Referring now to FIG. 1, depicted is an example system for performing color calibration using control tissue or calibration slides to automate detection, in accordance with one or more implementations. In brief overview, the system can include at least one data processing system 105, at least one network 110 (which may be the same as, or a part of, network 1526 described herein below in conjunction with FIG. 15), and at least one computing device 120. The data processing system 105 can include at least one calibration image obtainer 130, at least one intensity profile determiner 135, at least one slide image obtainer 140, at least one image presenter 145, and at least one score calculator 150. In some implementations, the data processing system 105 can include the data storage 115, and in some implementations, the data storage 115 can be external to the data processing system 105. For example, when the data storage 115 is external to the data processing system 105, the data processing system 105 (or the components thereof) can communicate with the data storage 115 via the network 110. In some implementations, the data processing system 105 can implement or perform any of the functionalities and operations discussed herein.

Each of the components (e.g., the data processing system 105, the network 110, the computing device 120, the calibration image obtainer 130, the intensity profile determiner 135, the slide image obtainer 140, the image presenter 145, and the score calculator 150, etc.) of the system 100 can be implemented using the hardware components or a combination of software with the hardware components of a computing system (e.g., server system 1500, client computing system 1514, any other computing system described herein, etc.) detailed herein in conjunction with FIG. 1500. Each of the components of the data processing system 105 can perform the functionalities detailed herein.

In further detail, the data processing system 105 can include at least one processor and a memory (e.g., a processing circuit). The memory can store processor-executable instructions that, when executed by processor, cause the processor to perform one or more of the operations described herein. The processor may include a microprocessor, an application-specific integrated circuit (ASIC), a field-programmable gate array (FPGA), etc., or combinations thereof. The memory may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, read-only memory (ROM), random-access memory (RAM), electrically erasable programmable ROM (EEPROM), erasable programmable ROM (EPROM), flash memory, optical media, or any other suitable memory from which the processor can read instructions. The instructions may include code from any suitable computer programming language. The data processing system 105 may include one or more computing devices or servers that can perform various functions as described herein. The data processing system 105 can include any or all of the components and perform any or all of the functions of the server system 1500 or the client computing system 1514 described herein below in conjunction with FIG. 15.

The network 110 can include computer networks such as the Internet, local, wide, metro, or other area networks, intranets, satellite networks, other computer networks such as voice or data mobile phone communication networks, and combinations thereof. In some implementations, the network 110 can be, be a part of, or include one or more aspects of the network 1526 described in connection with FIG. 15. The data processing system 105 of the system 100 can communicate via the network 110, for instance with at least one computing device 120. The network 110 can be any form of computer network that can relay information between the data processing system 105, the computing device 120, and in some implementations one or more external or third-party computing devices, such as web servers, among others. In some implementations, the network 110 can include the Internet and/or other types of data networks, such as a local area network (LAN), a wide area network (WAN), a cellular network, a satellite network, or other types of data networks.

The network 110 can also include any number of computing devices (e.g., computers, servers, routers, network switches, etc.) that are configured to receive and/or transmit data within the network 110. The network 110 can further include any number of hardwired and/or wireless connections. Any or all of the computing devices described herein (e.g., the data processing system 105, the computing device 120, the server system 1500, the client computing device 1514, etc.) can communicate wirelessly (e.g., via WiFi, cellular, radio, etc.) with a transceiver that is hardwired (e.g., via a fiber optic cable, a CAT5 cable, etc.) to other computing devices in the network 110. Any or all of the computing devices described herein (e.g., the data processing system 105, the computing device 120, the server system 1500, the client computing device 1514, etc.) can also communicate wirelessly with the computing devices of the network 110 via a proxy device (e.g., a router, network switch, or gateway).

The data storage 115 can be a database configured to store and/or maintain any of the information described herein. The data storage 115 can maintain one or more data structures, which may contain, index, or otherwise store each of the values, pluralities, sets, variables, vectors, thresholds, or any generated or determined information described herein. The data storage 115 can be accessed using one or more memory addresses, index values, or identifiers of any item, structure, or region maintained in the data storage 115. The data storage 115 can be accessed by the components of the data processing system 105, or any other computing device described herein, via the network 110. In some implementations, the data storage 115 can be internal to the data processing system 105. In some implementations, the data storage 115 can be external to the data processing system 105, and may be accessed via the network 110. The data storage 115 can be distributed across many different computer systems or storage elements, and may be accessed via the network 110 or a suitable computer bus interface.

The data processing system 105 can store, in one or more regions of the memory of the data processing system 105, or in the data storage 115, the results of any or all computations, determinations, selections, identifications, generations, constructions, or calculations in one or more data structures indexed or identified with appropriate values. Any or all values stored in the data storage 115 may be accessed by any computing device described herein, such as the data processing system 105, to perform any of the functionalities or functions described herein.

The computing device 120 can include at least one processor and a memory, e.g., a processing circuit. The memory can store processor-executable instructions that, when executed by processor, cause the processor to perform one or more of the operations described herein. The processor may include a microprocessor, an ASIC, an FPGA, etc., or combinations thereof. The memory may include, but is not limited to, electronic, optical, magnetic, or any other storage or transmission device capable of providing the processor with program instructions. The memory may further include a floppy disk, CD-ROM, DVD, magnetic disk, memory chip, ASIC, FPGA, ROM, RAM, EEPROM, EPROM, flash memory, optical media, or any other suitable memory from which the processor can read instructions. The instructions may include code from any suitable computer programming language. The computing device 120 can include one or more computing devices or servers that can perform various functions as described herein. The computing device 120 can include any or all of the components and perform any or all of the functions of the server system 1500 or the client computing system 1514 described herein below in conjunction with FIG. 15.

The scanner device 125 may be any type of scanner device capable of generating whole slide images (or portions thereof) of stained tissue in an IHC analysis process. The scanner device 125 may be used to generate any type of scan for any type of IHC stain, including membrane stains, nuclear stains, and cytoplasmic stains, among others. The scanner device 125 may be communicatively coupled to the data processing system 105 via the network 110, or another type of communications bus or input/output interface. The scanner device 125 can transmit any of the scanner images produced by scanning stained slides of tissue samples, for example, in response to one or more requests from the data processing system 105. In some implementations, the scanner device 125 may be in communication with one or more of the computing devices 120 (e.g., via the network 110, a communications bus, or an input/output interface, etc.). The scanner device 125 may provide any scanned images to the computing device 120, which may transmit the images to the data processing system 105 for further analysis or processing.

The scanner device 125 may associate any produced scanner images with a timestamp or time period, which corresponds to the date and time the images were captured by the scanner device 125. The scanner device 125 may produce images of slides at various magnification levels (e.g., 20×, 30×, 40×, 50×, 60×, etc.). The scanner device 125 may capture images at a predetermined resolution, for example, 0.13 microns/pixel (NA*0.95) or 0.24 microns/pixel (NA 0.75). Other magnifications and resolutions are also possible. The images produced by the scanner device 125 may be whole slide images, or portions of whole slide images. In some implementations, the scanner device 125 can store any produced images in the data storage 125, with any corresponding metadata (e.g., timestamps, identifiers of the scanner device 125, etc.). The data processing system 105 can utilize the images produced by the scanner device 125 to perform the operations described herein.

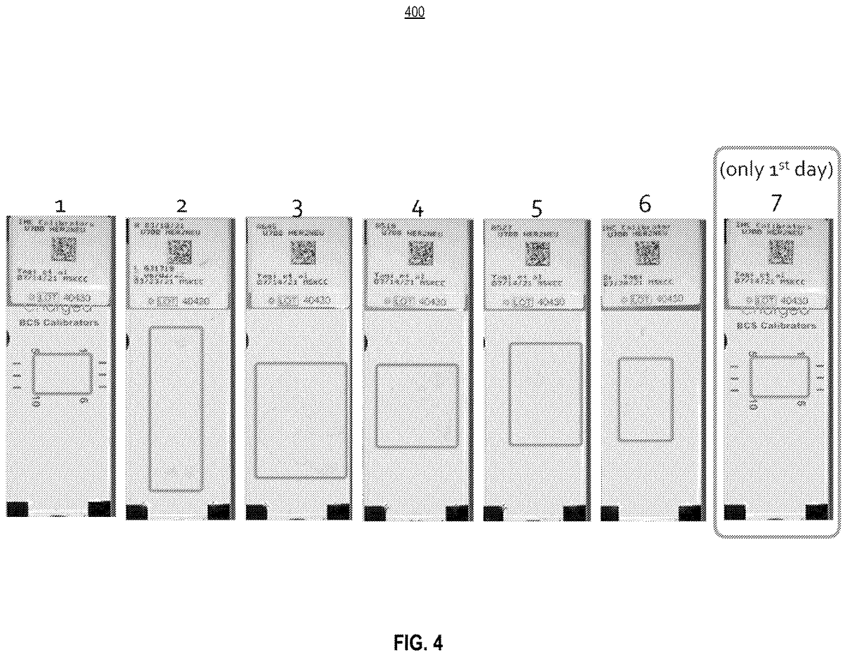

The calibration image obtainer 130 can receive one or more calibration images from the scanner device 125. Each of the one or more calibration images can depict a portion of a respective stained tissue sample having a known score value. Each of the calibration images are selected to correspond to a respective score value, and each can correspond to a respective slide that are scanned by the scanner device 125. Some examples of such calibration slides are shown in FIG. 4. In FIG. 4, certain slide images correspond to different days of the week. In some implementations, and as described in greater detail herein, the calibration processes described herein may be used to generate intensity profiles for the scanner device 125 over predetermined time periods. In such implementations, the calibration slides may be selected to correspond to certain days of the week, and may include tissue samples representative of a predetermined score (e.g., a 0, 1+, 2+, or 3+ HER2 score, etc.).

Referring back to FIG. 1, the data processing system 105 can be communicatively coupled with the scanner device 125, which can capture and provide the images to the calibration slides for further processing. In some implementations, the scanner device 125 can store the calibration image(s) in one or more data structures of the data storage 115. The one or more data structures containing the calibration images can be indexed by various parameters or characteristics of the slide, such as the attributes of the sample (e.g., tissue type, sample donor identifier, or other type of biomedical image identifier, etc.), the time that the calibration images were captured by the scanner device 125, and an identifier of the scanner device 125. The calibration image obtainer 130 can retrieve one or more images from the data storage 115, the scanner device 125, or the computing device(s) 120, and provide the calibration images to any of the components of the data processing system 105 for further processing.

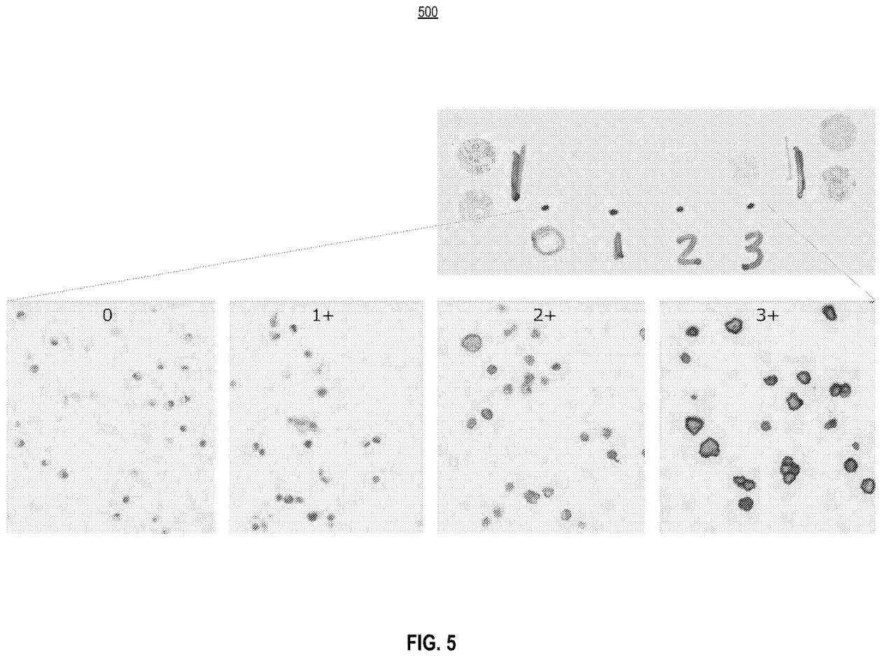

The calibration image obtainer 130 can identify, receive, or otherwise obtain one or more calibration images derived from a calibration tissue sample (e.g., selected to have a predetermined score value, such as a HER2 score). The calibration tissue sample may be sourced from a biopsy, a sample of living tissue, preserved tissue, or other biological matter. Referring briefly to FIG. 5, illustrated is an example calibration slide including different cases corresponding to different score values. As shown in FIG. 5, the slide includes tissues that are stained with IHC testing stain (e.g., DAB, etc.). Each of the sections of the slide correspond to a respective HER2 score, from left to right, 0, 1+, 2+, and 3+. Note that the staining color for each slide becomes a darker brown in the borderline and positive HER2 tissue samples. This example slide, or slides that include similar tissues, may be used to create calibration images used in the processes described herein. In some implementations, the calibration slides can include a nano particle-based calibrator (e.g., such as a calibrator manufactured by Boston Cell), a cell palette, at and least four typical HER2 breast cancer cases (e.g., each corresponding to a respective score from 0, 1+, 2+, and 3+). The calibration images can depict such calibration slides captured by the scanner device 125.

Referring back to FIG. 1, after receiving the calibration images, the calibration image obtainer 130 can store the calibration images in one or more data structures of the data storage 115. The calibration images obtained by the calibration image obtainer 130 can include, for example, any type of image file (e.g., JPEG, JPEG-2000, PNG, GIF, TIFF, etc.), including an SVS image file. The calibration image obtainer 130 can open, parse, or otherwise load the calibration images into working memory of the data processing system 105 (e.g., accessible by any of the components of the data processing system 105, etc.), using a slide parsing software. The slide parsing software may be any type of software capable of opening, modifying, or displaying any images captured using the scanner device 125. The calibration images can be stored in the data storage 115 in association with a timestamp corresponding to the time the calibration images were captured by the scanner device 125, and an identifier of the scanner device 125.

The intensity profile determiner 135 can determine an intensity profile for the scanner device 125. The intensity profile may be stored in the data storage 115, and can include a mapping between one or more score values (e.g., HER2 score values) and a respective color values. The intensity profile determiner 135 can determine the intensity profile based on the one or more calibration images corresponding to the scanner device 125, and can associate the intensity profile with a first time period (e.g., the time period associated with when the calibration images were created with the scanner device 125). To generate the intensity profile for a particular time period, the intensity profile determiner 135 can access the one or more calibration images obtained from the scanner device 125. As described above, each of the calibration images can be stored in association with a known score value (e.g., a known HER2 score value, as determined by a physician or another offline process). The intensity profile determiner 135 can determine the stain profile by estimating the staining intensity for each of the control tissues or calibration slides used to generate the calibration images (e.g., each section of the WSI corresponding to the control tissues or calibration slides having HER2 scores of 0, 1+, 2+, and 3+).

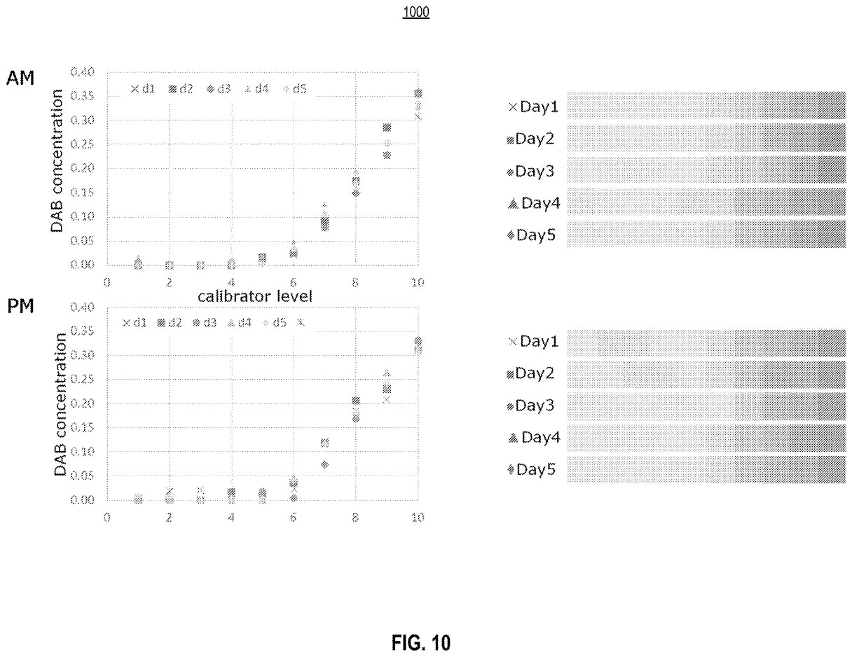

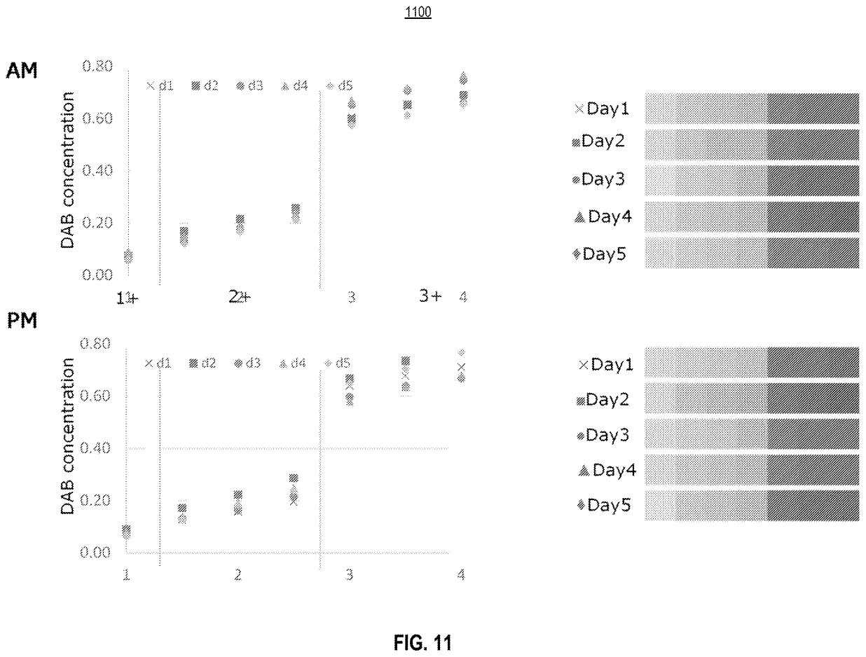

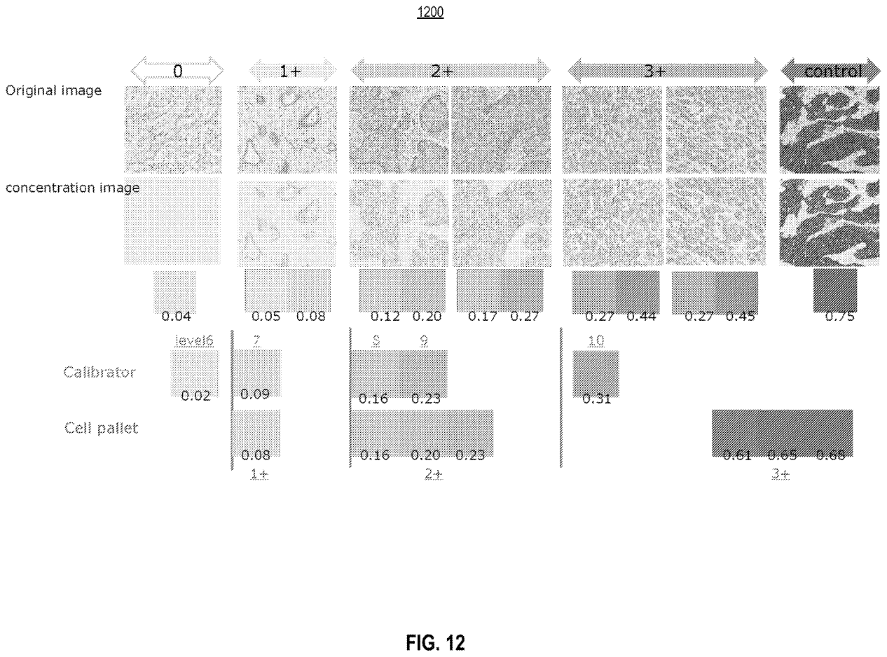

In some implementations, the intensity profile determiner 135 can determine the intensity profile for the scanner device 125 over a multiple day period. For example, multiple different calibration slides may be utilized to generate corresponding daily color bars for the scanner device 125. The color bars may indicate a mapping between HER2 scores and intensity levels in slide images. In some implementations, the intensity profile may be populated with a number of color mappings, each of which correspond to the time period (e.g., morning or evening) and day that the respective calibration slide was scanned. Examples of these color bars, which may be calculated based the estimated DAB concentration in the calibration slides, is shown in FIG. 10. Corresponding slide images for an example cell pallet, which may be included in the calibration slides, is shown in FIG. 11 in connection with respective color bars generated from the cell pallet images. Additionally, FIG. 12 shows a comparison between intensity values of a calibrator, a cell pallet, and tissue samples corresponding to 0, 1+, 2+, 3+, and control HER2 scores.

Referring back to FIG. 1, in some implementations, the intensity profile determiner 135 can perform a color calibration process. The color calibration process can be performed, for example, by utilizing one or more portions of the WSI dedicated to the calibrator (e.g., which may include regions having a predetermined colors or intensities). The color calibration technique can be used, for example, to generate a color calibration matrix, which may be applied to slide images, such as the calibration images obtained by the calibration image obtainer 130. In some implementations, a stain calibration process may also be performed. The color calibration technique may be the color calibration technique described in Bautista P A, Hashimoto N, Yagi Y. Color standardization in whole slide imaging using a color calibration slide. J Pathol Inform 2014; 5(1)4, the contents of which is incorporated by reference herein in its entirety. The color calibration matrix M may be applied to the red, green and blue (R, G, and B; or sometimes “RGB”) values of each pixel in the image to which the calibration matrix is applied. An example of this operation is provided below, where the R, G, and B values of each pixel i are modified according to the matrix M:

$$( \begin{matrix}{R}_{i}^{′} \\ {G}_{i}^{′} \\ {B}_{i}^{′}\end{matrix} ) = M ( \begin{matrix}{R}_{i} \\ {G}_{i} \\ {B}_{i}\end{matrix} ) = ( \begin{matrix}{a}_{11} & {a}_{12} & {a}_{13} \\ {a}_{21} & {a}_{22} & {a}_{23} \\ {a}_{31} & {a}_{32} & {a}_{33}\end{matrix} ) ( \begin{matrix}{R}_{i} \\ {G}_{i} \\ {B}_{i}\end{matrix} )$$

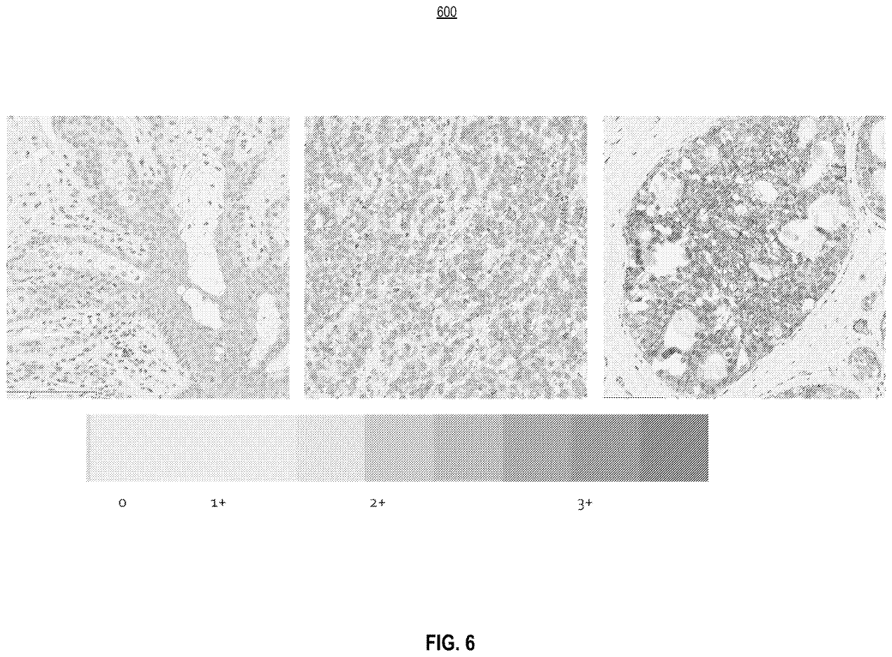

After applying the calibration matrix to the slide image (e.g., a calibrator image or an unscored image, etc.), the intensity profile determiner 135 can determine the intensity profile for the slide image using the processes described herein. The generated intensity profile can be stored in association with an identifier of the scanner device 125 used to generate the intensity profile. An example image showing a color mapping of an intensity profile that is generated from corresponding calibration images is shown in FIG. 6. As shown in FIG. 6, the intensity profile includes a color mapping between different color intensities and corresponding HER2 score values. Each of the slide images, from left to right, depict a 0-1+ HER2 score, a 2+ HER2 score, and a 3+ HER2 score.

Referring back to FIG. 1, once the intensity profile has been generated for the scanner device 125, unscored slide images can be analyzed using the intensity profile. The slide image obtainer 140 can receive an unscored slide image depicting a portion of stained tissue, for example, a sample from a breast cancer biopsy. The unscored slide image can be captured by and received from the scanner device 125 during the first time period. The slide image obtainer 140 can obtain the unscored slide image, for example, by sending a request to the scanner device 125. In some implementations, the scanner device 125 can store the unscored slide image(s) in one or more data structures of the data storage 115. The one or more data structures containing the unscored slide images can be indexed by various parameters or characteristics of the slide, such as the attributes of the sample (e.g., tissue type, sample donor identifier, or other type of biomedical image identifier, etc.), the time that the calibration images were captured by the scanner device 125, and an identifier of the scanner device 125. The slide image obtainer 140 can retrieve one or more unscored slide images from the data storage 115, the scanner device 125, or the computing device(s) 120, and provide the unscored slide images to any of the components of the data processing system 105 for further processing. The unscored slide images can depict a portion of a respective stained tissue sample with an unknown score value. The stained tissue sample may be stained with, for example, a DAB solution.

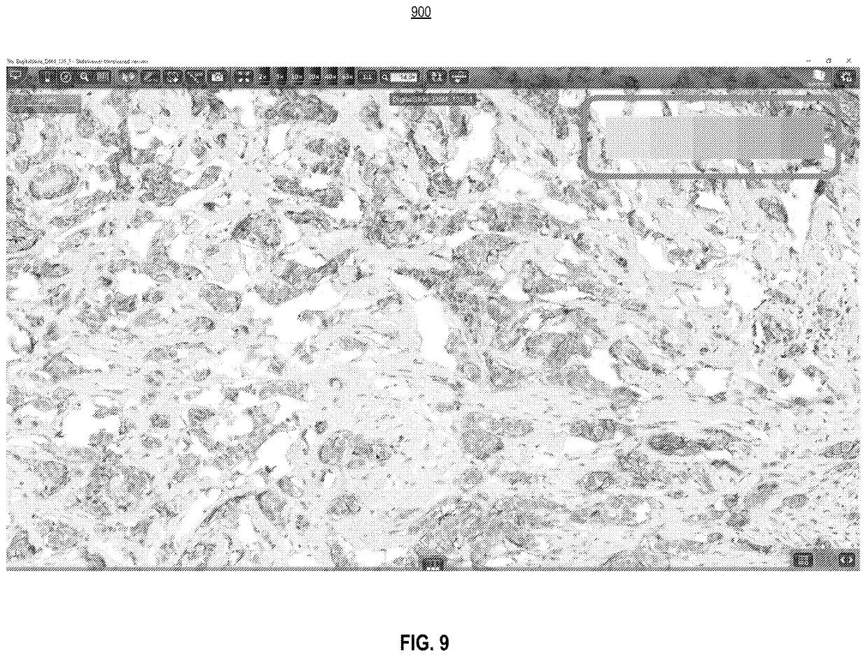

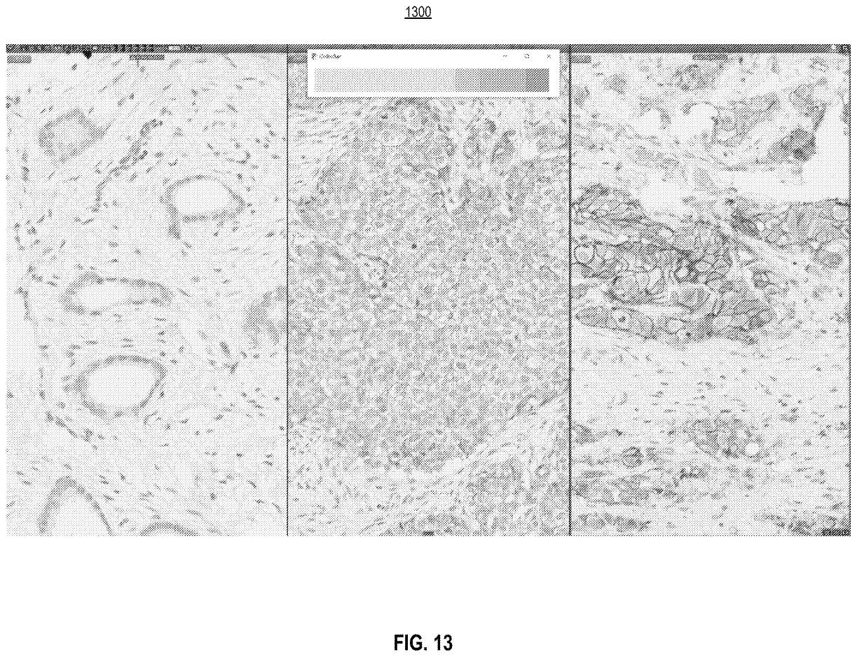

The image presenter 145 can present, in a graphical user interface of an application executing on the data processing system 105, the unscored slide image with the intensity profile corresponding to the first time period. Upon receiving the unscored slide image, the image presenter 145 can display the unscored slide image in a slide viewer application. The image presenter 145 can present the unscored slide image in a user interface with the color mapping (e.g., the color bars) of the intensity profile, for example, as shown the user interfaces shown in FIGS. 9 and 13. In FIG. 9, the color bar of the intensity profile is displayed in connection with a single slide image. As shown, the color bar of the intensity profile can be displayed as an overlay in the same display window as the unscored slide image. In FIG. 13, the color bar of the intensity profile is displayed in connection with several slide images (e.g., shown here as three separate slide images). In some implementations, the user interface of FIG. 13 may be used to display one or more separate portions of the same WSI, for example, at varying magnification levels. This allows for a physician to accuracy assess the differences between the intensity colors (and the associated HER2 score values) and the portions WSI of an unscored sample.

Referring back to FIG. 1, the techniques described herein may be used to generate an intensity profile for any scanner device 125, for any predetermined period of time. As such, the techniques described herein may be utilized to analyze unscored slide images more accurately, my displaying the color bars of the determined intensity profile with the unscored slide images (e.g., which may be whole slide images or portions thereof). To do so, the image presenter 145 can receive and unscored slide image generated by a scanner device 125 during an identified time period. Identifiers of the time period and the scanner device 125 may be received with the unscored slide image, for example, in response to a request or as part of a data package. In some implementations, the slide metadata (e.g., the scanner device 125 identifier and the time period identifier) may be received separately from the unscored slide image. Using the slide metadata, the image presenter 145 can identify an intensity profile (e.g., stored in the data storage 115) determined for the scanner device 125 for the identified time period, and present the unscored slide image in the application with the identified intensity profile, as shown in FIGS. 9 and 13.

In addition to utilizing the intensity profiles to display generated color bars with unscored whole slide images, the score determiner 150 can determine a score for the unscored slide image based on the intensity profile. To do so, the score determiner 150 can determine one or more color thresholds corresponding to each of the score values (e.g., each HER2 score value). The color thresholds may be defined, for example, based on the color mapping indicated in the intensity profile. Each color intensity value can indicate a lower bound threshold, and any intensity values (e.g., pixel intensity values) that are greater in value of the threshold (e.g., up to the next threshold value) indicate the corresponding score value. Based on the color thresholds in the slide image, the score determiner 150 scan through each of the pixels in the slide image to determine an average stain intensity value. The average stain intensity value may be compared to the mappings between intensity values and corresponding HER2 scores. The appropriate HER2 score indicated in the mapping can then be selected based on the average stain intensity, and the score value can be associated with the unscored slide image in the data storage 115.

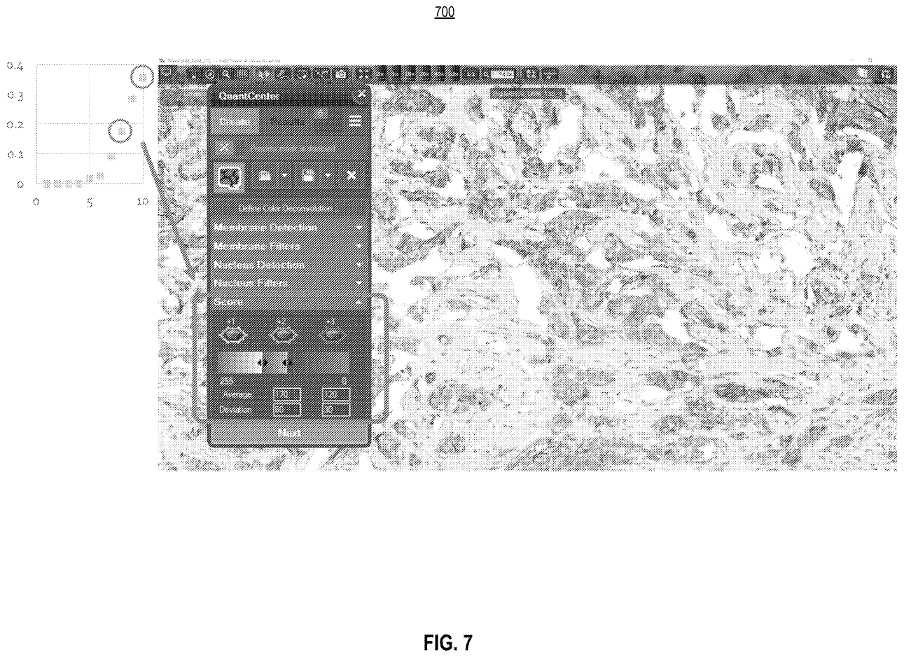

The application executed by the image presenter 145 may allow for modification of these thresholds, as shown in FIG. 7. In FIG. 7, the user may modify these thresholds (e.g., according to detected stain intensity), and set corresponding annotation colors for each score value. These annotation colors may be represented as a gradient, which may fade in intensity (e.g., dark yellow to light yellow) based on the intensity of the HER2 score (e.g., 1+ scores that are relative high intensity may be assigned a light yellow color, while low intensity 1+ scores may be assigned a dark yellow color, as shown). As described herein, the intensity mapping may be selected at least in part based on an estimated DAB concentration detected in the slide image.

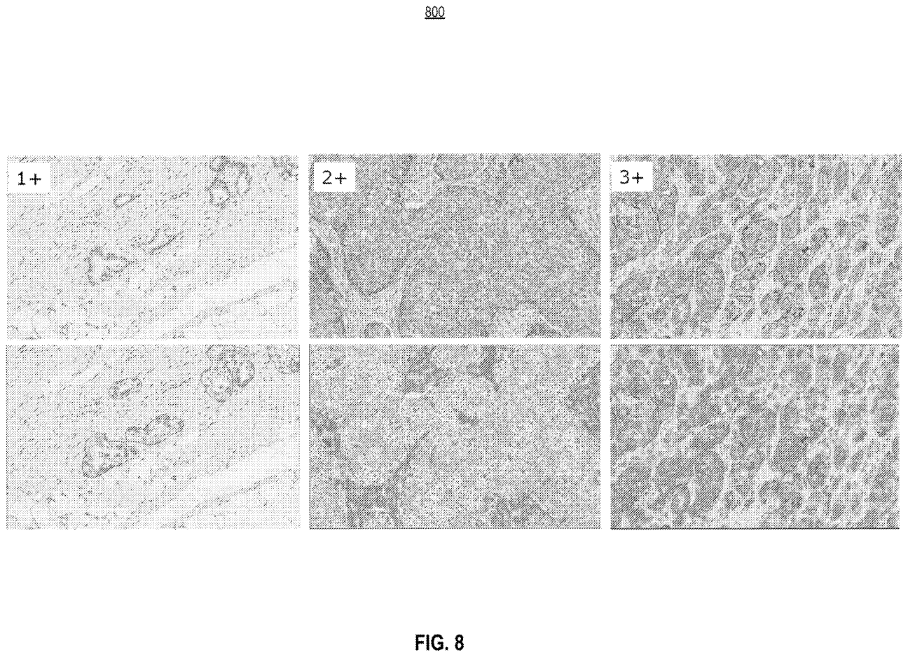

Referring back to FIG. 1, after selecting these colors, the image presenter 145 can scan through each pixel in the slide image, retrieve the stain intensity value (e.g., a pixel color) for each pixel, and assign the annotation color to that pixel based on the annotation mapping. A result of this process is shown in FIG. 8. As shown in FIG. 8, the regions of the unscored slide image that correspond to the intensity values indicated in the annotation mappings between have been annotated with the corresponding annotation color. Three cases are shown, with the 1+ case being mostly unannotated, but with some small portions annotated in yellow (indicated a 1+ case), with the 2+ case being largely annotated in yellow and orange, and with the 3+ case mostly annotated in red. In some implementations, a user of the application can turn annotations on or off using one or more interactive user interface elements (not shown).

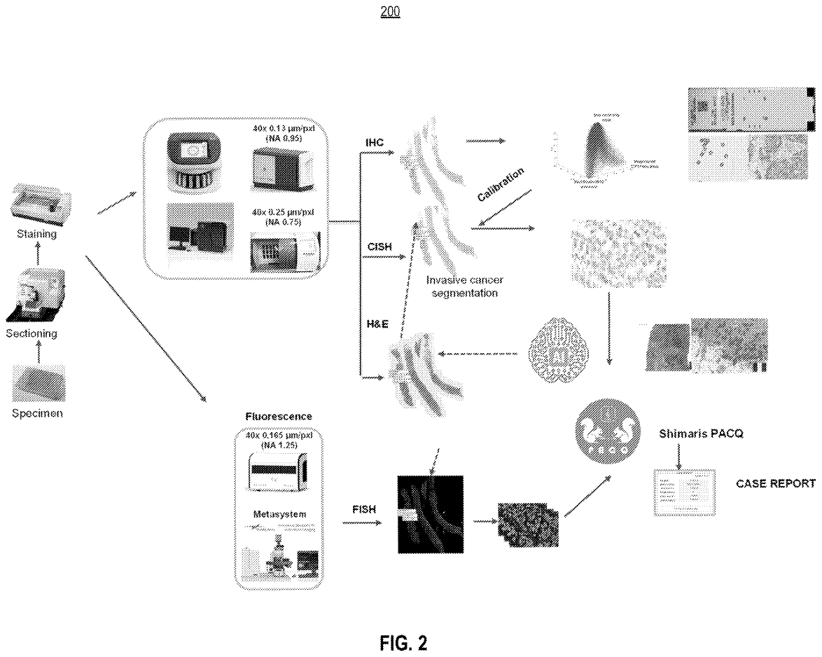

Referring to FIG. 2, illustrated is an example system architecture diagram 200 showing a process for processing stained slide images using the color calibration techniques described herein, in accordance with one or more implementations. This example architecture may be used to carry out the processes and techniques described herein. As shown, samples are first collected (e.g., from a biopsy), and slides are created and stained. The stained slides are then provided to various scanning devices, which produce images for the slides (e.g., whole slide images). The slide images (which may be unscored slide images or calibration images) may then be used in the techniques described herein, or as part of various other processes, including chromogenic in situ hybridization (CISH), to fluorescence in situ hybridization (FISH), IHC testing, or H&E testing, among others. The control tissue or calibration slide-based scoring and calibration techniques described herein may be utilized in any number of these techniques, or in processes involving these techniques. As shown, various other processes may be used to aid in cancer detection and treatment, including artificial intelligence-based processes or segmentation-based processes. The scoring information for a particular slide image may be included in a case report for the patient associated with the slide image.

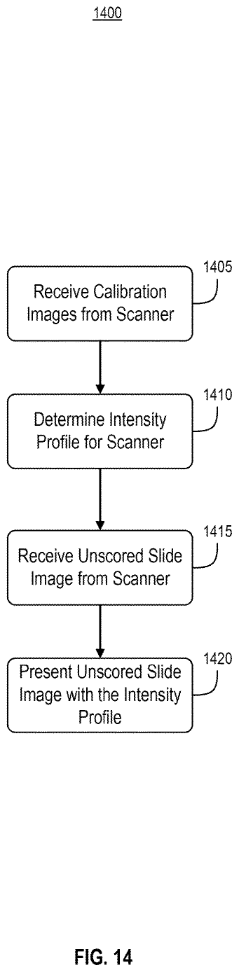

Referring now to FIG. 14, depicted is a flow diagram of an example method 1400 of the analysis and quality control of whole slide images in accordance an illustrative embodiment. The method 1400 can be performed, for example, by any computing device described herein, including the data processing system 105. In brief overview, in performing the method 1400, the data processing system (e.g., the data processing system 105, etc.) can receive calibration images from a scanner device (e.g., the scanner device 125) (STEP 1405), determine an intensity profile for the scanner device (STEP 1410), receive an unscored slide image from the scanner device (STEP 1415), and present the unscored slide image with the intensity profile (STEP 1420).

In further detail of the method 1400, at step 1405, the data processing system can receive calibration images from a scanner device (e.g., the scanner device 125). Each of the one or more calibration images can depict a portion of a respective stained tissue sample having a known score value. Each of the calibration images are selected to correspond to a respective score value, and each can correspond to a respective slide that are scanned by the scanner device. In some implementations, the scanner device can store the calibration image(s) in one or more data structures of a data storage (e.g., the data storage 115) of the data processing system. The one or more data structures containing the calibration images can be indexed by various parameters or characteristics of the slide, such as the attributes of the sample (e.g., tissue type, sample donor identifier, or other type of biomedical image identifier, etc.), the time that the calibration images were captured by the scanner device, and an identifier of the scanner device. The data processing system can retrieve one or more images from the data storage, the scanner device, or other computing device(s) (e.g., the computing device(s) 120), and provide the calibration images to any of the components of the data processing system for further processing.

The data processing system can store the calibration images in one or more data structures of the data storage. The calibration images obtained by the data processing system can include, for example, any type of image file (e.g., JPEG, JPEG-2000, PNG, GIF, TIFF, etc.), including an SVS image file. The data processing system can open, parse, or otherwise load the calibration images into working memory of the data processing system (e.g., accessible by any of the components of the data processing system, etc.), using a slide parsing software. The slide parsing software may be any type of software capable of opening, modifying, or displaying any images captured using the scanner device. The calibration images can be stored in the data storage in association with a timestamp corresponding to the time the calibration images were captured by the scanner device, and an identifier of the scanner device.

At step 1410, the data processing system can determine a correction function (sometimes referred to as an intensity profile) for the scanner device. The intensity profile may be stored in the data storage, and can be used to generate a mapping between one or more score values (e.g., HER2 score values) and a respective color values. In some implementations, the intensity profile can include one or more data structures that store a mapping or relationship between one or more score values (e.g., HER2 score values) and a respective color values. The data processing system can determine the intensity profile based on the one or more calibration images corresponding to the scanner device, and can associate the intensity profile with a first time period (e.g., the time period associated with when the calibration images were created with the scanner device). To generate the intensity profile for a particular time period, the data processing system can access the one or more calibration images obtained from the scanner device. As described above, each of the calibration images can be stored in association with a known score value (e.g., a known HER2 score value, as determined by a physician or another offline process). The data processing system can determine the stain profile by estimating the staining intensity for the calibration slide(s) or each of the control tissues of the calibration images (e.g., each section of the WSI corresponding to the calibration slide(s) or the control tissues having HER2 scores of 0, 1+, 2+, and 3+).

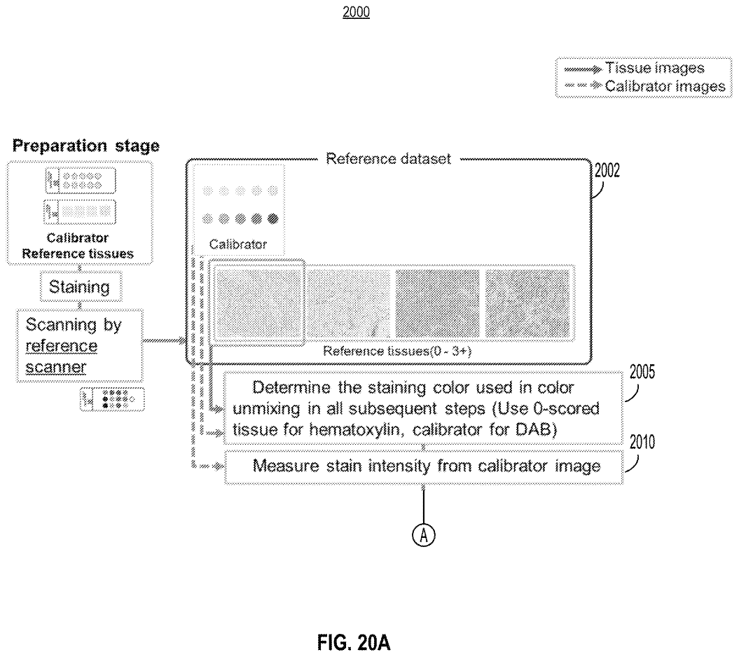

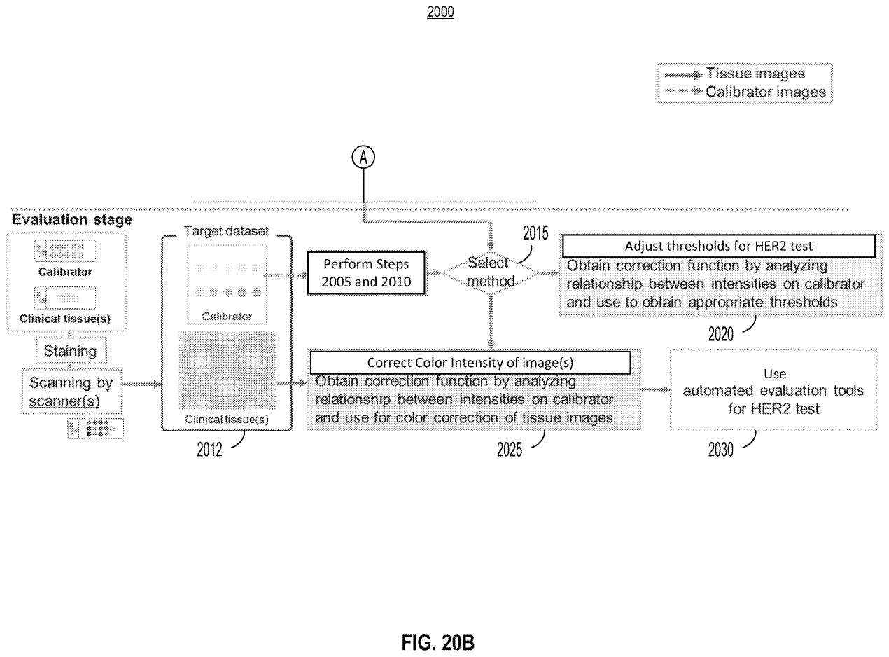

In some implementations, the data processing system can determine the intensity profile for the scanner device over a multiple day period. For example, multiple different calibration slides may be utilized to generate corresponding daily color bars for the scanner device. The color bars may indicate a mapping between HER2 scores and intensity levels in slide images. In some implementations, the intensity profile may be populated with a number of color mappings, each of which correspond to the time period (e.g., morning or evening) and day that the respective calibration slide was scanned. In some implementations, the data processing system can perform a color calibration process. The color calibration process can be performed, for example, by utilizing one or more portions of the WSI dedicated to the calibrator (e.g., which may include regions having a predetermined colors or intensities). The color calibration technique can be used, for example, to generate a color calibration matrix, which may be applied to slide images, such as the calibration images obtained by the data processing system. In some implementations, a stain calibration process may also be performed. After applying the calibration matrix to the slide image (e.g., a calibrator image or an unscored image, etc.), the data processing system can determine the intensity profile for the slide image using the processes described herein. The generated intensity profile can be stored in association with an identifier of the scanner device used to generate the intensity profile. In some implementations, the data processing system can determine the correction function (and any values that may be included in the intensity profile) for the scanner using the techniques described in connection with FIGS. 20A, 20B, 32, 33, 34, and 35, among other techniques described herein.

At step 1415, the data processing system can receive an unscored slide image from the scanner device (STEP 1415). The data processing system can receive an unscored slide image depicting a portion of stained tissue, for example, a sample from a breast cancer biopsy. The unscored slide image can be captured by and received from the scanner device during the first time period. The data processing system can obtain the unscored slide image, for example, by sending a request to the scanner device. In some implementations, the scanner device can store the unscored slide image(s) in one or more data structures of the data storage. The one or more data structures containing the unscored slide images can be indexed by various parameters or characteristics of the slide, such as the attributes of the sample (e.g., tissue type, sample donor identifier, or other type of biomedical image identifier, etc.), the time that the calibration images were captured by the scanner device, and an identifier of the scanner device. The data processing system can retrieve one or more unscored slide images from the data storage, the scanner device, or the other computing device(s) in communication with the data processing system, and provide the unscored slide images to any of the components of the data processing system for further processing. The unscored slide images can depict a portion of a respective stained tissue sample with an unknown score value. The stained tissue sample may be stained with, for example, a DAB solution.

At step 1420, the data processing system can present the unscored slide image with the intensity profile. Upon receiving the unscored slide image, the data processing system can display the unscored slide image in a slide viewer application. The data processing system can present the unscored slide image in a user interface with the color mapping (e.g., the color bars) of the intensity profile. The techniques described herein may be used to generate an intensity profile for any scanner device, for any predetermined period of time. As such, the techniques described herein may be utilized to analyze unscored slide images more accurately, my displaying the color bars of the determined intensity profile with the unscored slide images (e.g., which may be whole slide images or portions thereof). To do so, the data processing system can receive and unscored slide image generated by a scanner device during an identified time period. Identifiers of the time period and the scanner device may be received with the unscored slide image, for example, in response to a request or as part of a data package. In some implementations, the slide metadata (e.g., the scanner device identifier and the time period identifier) may be received separately from the unscored slide image. Using the slide metadata, the data processing system can identify an intensity profile (e.g., including a correction function stored in the data storage) determined for the scanner device for the identified time period, and present the unscored slide image in the application with the identified intensity profile, as shown in FIGS. 9 and 13. In some implementations, the data processing system can execute the correction function to determine a score for the unscored slide image (e.g., a HER2 score), and can display the score in a graphical user interface.

The foregoing techniques may be utilized for performing color calibration using control tissue or calibration slides to automate detection. In some implementations, the data processing system can perform automatic correction of colors present in the unscored slide image using the correction function, using the techniques described in connection with FIGS. 20A, 20B, 32, 33, 34, and 35, among other techniques described herein. A color of whole slide images may strongly depend on the scanner system used to prepare the slide images. Slide preparation can include staining the tissue. Color differences can occur even if following standard staining protocols and small differences are important in many of IHC tests which evaluate stain intensity. Staining quality and characteristics need to be understood at each laboratory, such that the color differences can be compensated for to enable appropriate assessment. The present techniques provide IHC calibrator tools for whole slide image based assessment. Experimental results are provided that evaluate the present techniques to see if they are helpful in the process of image-based manual/automated IHC evaluations.

The experimental results were created using six slides each containing nano particle-based calibrator, cell palette, and four typical HER2 0-3+ breast cancer cases in our institution as a dataset. Twenty-eight datasets were obtained in 10 weeks. The calibrator is used for staining quality assessment, and the cell palette is used to assist visual assessment. We estimated staining intensity from WSI. Intensity score color-bars were created based on estimated staining intensity to visualize staining variation and to compare performance of each tool. Furthermore, threshold values used for automatic HER2 score assessment to be confirmed if correct score is obtained with typical cases.

Differences in color and staining intensity were visualized well and automatic score assessment were accurate. Intensity score color-bars were shared with the corresponding labs to demonstrate staining condition (stability). Pathologists may use the intensity score color-bars visually for manual HER2 assessment. Since 3+ cells on cell palette are darker than typical 3+ case of our institution, percentage of positive cells tends to be underestimated. On the other hand, since the calibrator is used for different from original purpose, correspondence with tissues is not always linear, and make correction in consideration of these matters is desired.

The present techniques help to measure (or quantify) the differences in color and staining intensity. It will make us possible to evaluate any WSIs of IHC score accurately. Also, even if there are differences, the present techniques may be used to assess either manually or automatically.

Various operations described herein can be implemented on computer systems. FIG. 15 shows a simplified block diagram of a representative server system 1500, client computer system 1514, and network 1526 usable to implement certain embodiments of the present disclosure. In various embodiments, server system 1500 or similar systems can implement services or servers described herein or portions thereof. Client computer system 1514 or similar systems can implement clients described herein. The system 100 described herein can be similar to the server system 1500. Server system 1500 can have a modular design that incorporates a number of modules 1502 (e.g., blades in a blade server embodiment); while two modules 1502 are shown, any number can be provided. Each module 1502 can include processing unit(s) 1504 and local storage 1506.

Processing unit(s) 1504 can include a single processor, which can have one or more cores, or multiple processors. In some embodiments, processing unit(s) 1504 can include a general-purpose primary processor as well as one or more special-purpose co-processors such as graphics processors, digital signal processors, or the like. In some embodiments, some or all processing units 1504 can be implemented using customized circuits, such as application specific integrated circuits (ASICs) or field programmable gate arrays (FPGAs). In some embodiments, such integrated circuits execute instructions that are stored on the circuit itself. In other embodiments, processing unit(s) 1504 can execute instructions stored in local storage 1506. Any type of processors in any combination can be included in processing unit(s) 1504.

Local storage 1506 can include volatile storage media (e.g., DRAM, SRAM, SDRAM, or the like) and/or non-volatile storage media (e.g., magnetic or optical disk, flash memory, or the like). Storage media incorporated in local storage 1506 can be fixed, removable, or upgradeable as desired. Local storage 1506 can be physically or logically divided into various subunits such as a system memory, a read-only memory (ROM), and a permanent storage device. The system memory can be a read-and-write memory device or a volatile read-and-write memory, such as dynamic random-access memory. The system memory can store some or all of the instructions and data that processing unit(s) 1504 need at runtime. The ROM can store static data and instructions that are needed by processing unit(s) 1504. The permanent storage device can be a non-volatile read-and-write memory device that can store instructions and data even when module 1502 is powered down. The term “storage medium” as used herein includes any medium in which data can be stored indefinitely (subject to overwriting, electrical disturbance, power loss, or the like) and does not include carrier waves and transitory electronic signals propagating wirelessly or over wired connections.

In some embodiments, local storage 1506 can store one or more software programs to be executed by processing unit(s) 1504, such as an operating system and/or programs implementing various server functions such as functions of the system 100 of FIG. 1 or any other system described herein, or any other server(s) associated with system 100 or any other system described herein.

“Software” refers generally to sequences of instructions that, when executed by processing unit(s) 1504 cause server system 1500 (or portions thereof) to perform various operations, thus defining one or more specific machine embodiments that execute and perform the operations of the software programs. The instructions can be stored as firmware residing in read-only memory and/or program code stored in non-volatile storage media that can be read into volatile working memory for execution by processing unit(s) 1504. Software can be implemented as a single program or a collection of separate programs or program modules that interact as desired. From local storage 1506 (or non-local storage described below), processing unit(s) 1504 can retrieve program instructions to execute and data to process in order to execute various operations described above.

In some server systems 1500, multiple modules 1502 can be interconnected via a bus or other interconnect 1508, forming a local area network that supports communication between modules 1502 and other components of server system 1500. Interconnect 1508 can be implemented using various technologies including server racks, hubs, routers, etc.

A wide area network (WAN) interface 1510 can provide data communication capability between the local area network (interconnect 1508) and the network 1526, such as the Internet. Technologies can be used, including wired (e.g., Ethernet, IEEE 802.3 standards) and/or wireless technologies (e.g., Wi-Fi, IEEE 802.11 standards).

In some embodiments, local storage 1506 is intended to provide working memory for processing unit(s) 1504, providing fast access to programs and/or data to be processed while reducing traffic on interconnect 1508. Storage for larger quantities of data can be provided on the local area network by one or more mass storage subsystems 1512 that can be connected to interconnect 1508. Mass storage subsystem 1512 can be based on magnetic, optical, semiconductor, or other data storage media. Direct attached storage, storage area networks, network-attached storage, and the like can be used. Any data stores or other collections of data described herein as being produced, consumed, or maintained by a service or server can be stored in mass storage subsystem 1512. In some embodiments, additional data storage resources may be accessible via WAN interface 1510 (potentially with increased latency).

Server system 1500 can operate in response to requests received via WAN interface 1510. For example, one of modules 1502 can implement a supervisory function and assign discrete tasks to other modules 1502 in response to received requests. Work allocation techniques can be used. As requests are processed, results can be returned to the requester via WAN interface 1510. Such operation can generally be automated. Further, in some embodiments, WAN interface 1510 can connect multiple server systems 1500 to each other, providing scalable systems capable of managing high volumes of activity. Other techniques for managing server systems and server farms (collections of server systems that cooperate) can be used, including dynamic resource allocation and reallocation.

Server system 1500 can interact with various user-owned or user-operated devices via a wide-area network such as the Internet. An example of a user-operated device is shown in FIG. 15 as client computing system 1514. Client computing system 1514 can be implemented, for example, as a consumer device such as a smartphone, other mobile phone, tablet computer, wearable computing device (e.g., smart watch, eyeglasses), desktop computer, laptop computer, and so on.

For example, client computing system 1514 can communicate via WAN interface 1510. Client computing system 1514 can include computer components such as processing unit(s) 1516, storage device 1518, network interface 1520, user input device 1522, and user output device 1524. Client computing system 1514 can be a computing device implemented in a variety of form factors, such as a desktop computer, laptop computer, tablet computer, smartphone, other mobile computing device, wearable computing device, or the like.

Processor 1516 and storage device 1518 can be similar to processing unit(s) 1504 and local storage 1506 described above. Suitable devices can be selected based on the demands to be placed on client computing system 1514; for example, client computing system 1514 can be implemented as a “thin” client with limited processing capability or as a high-powered computing device. Client computing system 1514 can be provisioned with program code executable by processing unit(s) 1516 to enable various interactions with server system 1500.

Network interface 1520 can provide a connection to the network 1526, such as a wide area network (e.g., the Internet) to which WAN interface 1510 of server system 1500 is also connected. In various embodiments, network interface 1520 can include a wired interface (e.g., Ethernet) and/or a wireless interface implementing various RF data communication standards such as Wi-Fi, Bluetooth, or cellular data network standards (e.g., 3G, 4G, LTE, etc.).

User input device 1522 can include any device (or devices) via which a user can provide signals to client computing system 1514; client computing system 1514 can interpret the signals as indicative of particular user requests or information. In various embodiments, user input device 1522 can include any or all of a keyboard, touch pad, touch screen, mouse or other pointing device, scroll wheel, click wheel, dial, button, switch, keypad, microphone, and so on.

User output device 1524 can include any device via which client computing system 1514 can provide information to a user. For example, user output device 1524 can include a display to display images generated by or delivered to client computing system 1514. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). Some embodiments can include a device such as a touchscreen that functions as both input and output device. In some embodiments, other user output devices 1524 can be provided in addition to or instead of a display. Examples include indicator lights, speakers, tactile “display” devices, printers, and so on.

Some embodiments include electronic components, such as microprocessors, storage, and memory that store computer program instructions in a computer readable storage medium. Many of the features described in this specification can be implemented as processes that are specified as a set of program instructions encoded on a computer readable storage medium. When these program instructions are executed by one or more processing units, they cause the processing unit(s) to perform various operations indicated in the program instructions. Examples of program instructions or computer code include machine code, such as is produced by a compiler, and files including higher-level code that are executed by a computer, an electronic component, or a microprocessor using an interpreter. Through suitable programming, processing unit(s) 1504 and 1516 can provide various functionality for server system 1500 and client computing system 1514, including any of the functionality described herein as being performed by a server or client, or other functionality.

It will be appreciated that server system 1500 and client computing system 1514 are illustrative and that variations and modifications are possible. Computer systems used in connection with embodiments of the present disclosure can have other capabilities not specifically described here. Further, while server system 1500 and client computing system 1514 are described with reference to particular blocks, it is to be understood that these blocks are defined for convenience of description and are not intended to imply a particular physical arrangement of component parts. For instance, different blocks can be but need not be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Embodiments of the present disclosure can be realized in a variety of apparatus including electronic devices implemented using any combination of circuitry and software.

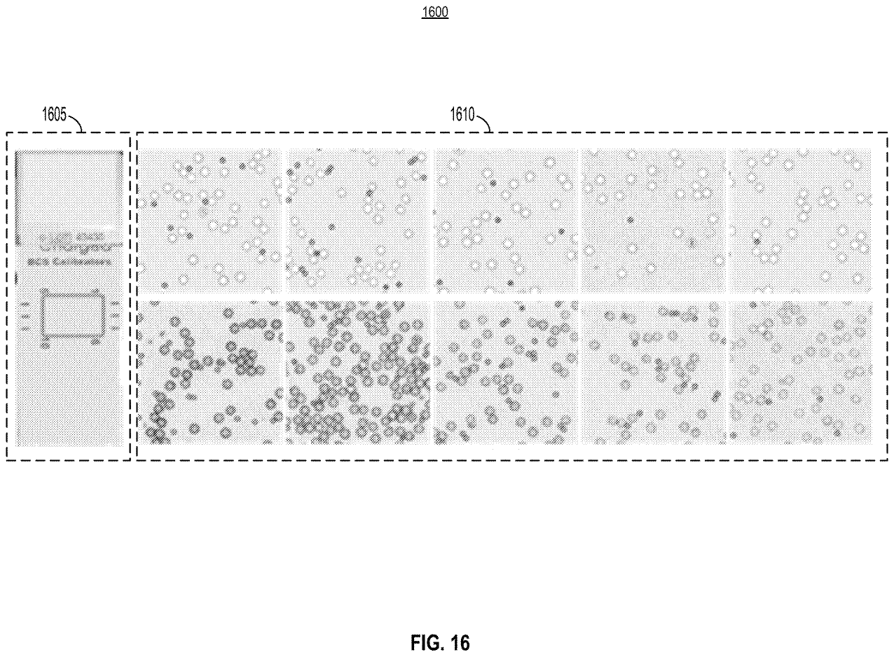

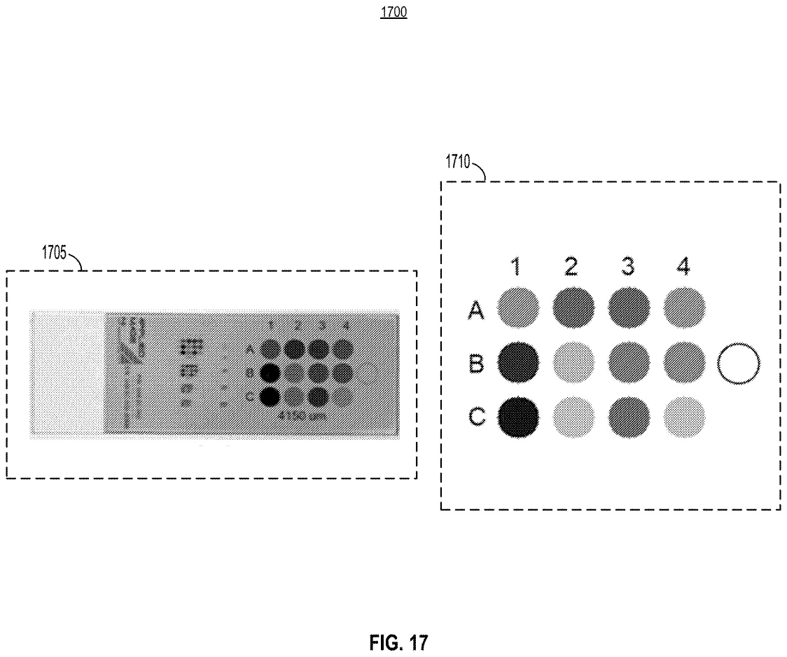

Further approaches to the preparation and evaluation of the various calibration approaches are provided herein, in the context of techniques that may be implemented by the system 100 of FIG. 1. The various techniques that follow are described in connection with example experimental results, which provided to show non-limiting example performance of the techniques described herein using control and clinical tissue samples. The techniques that follow may utilize the various IHC calibrator slides described herein, including the calibrator slide shown in FIG. 16 and the color reference slide shown in FIG. 17.

Referring to FIG. 16, depicted are the views 1605 and 1610 of an example calibration slide including various stain intensities, in accordance with one or more implementations. The view 1605 shows an example slide image of an IHC calibrator slide for HER2. The view 1610 shows an example whole slide image of the IHC calibrator slide for HER2 scanned at 0.23 μm per pixel resolution and displayed at 20× magnification, level 1 to 5 (top right to left), 6 to 10 (bottom right to left). The IHC calibrator slide for HER2 may be utilized in connection with any of the techniques described herein, including the techniques implemented by the data processing system 105 shown in FIG. 1.

The IHC calibrator slide for HER2 can include microbeads coated with different amounts of peptide concentration. The DAB color intensities of microbeads in the calibrator image correlate with those of the tissue sample images. The DAB color is obtained from the color of microbeads, and the intensity characteristics are derived from the intensities of the different levels of microbeads. Color intensities obtained from the calibrator images are used to standardize tissue images. We confirmed that the proposed method can calibrate color and intensity and classify HER2 status with less variability between datasets. The evaluation has been performed using a single scanner to focus on variation of staining. In some implementations, the various color calibration techniques described herein can be utilized to determine colors of different portions of different types of tissues, in any type of whole slide image (e.g., CISH, FISH, H&E, etc.). In some implementations, a calibrator tissue can be utilized to determine the color of nuclei, CEP17, or HER2. In some implementations, calibrator tissues may be utilized in lieu of an IHC calibrator slide including microbeads, for example, for techniques utilizing CISH, FISH, or H&E.

Referring to FIG. 17, depicted are views 1705 and 1710 of an example color chart slide including various reference color, in accordance with one or more implementations. The view 1705 shows an example slide image of the color chart slide. The view 1710 shows a close-up view of the reference colors present on the color chart slide. The color chart slide may be utilized in connection with any of the techniques described herein, including the techniques implemented by the data processing system 105 shown in FIG. 1.

In one non-limiting example experiment, a calibrator slide and tissue of breast cancer slides were stained together at different dates continuously. The stained slides and the color chart slide were digitized by two WSI scanners. The various color calibration implemented by the data processing system 105 were utilized to address both variations due to staining and scanning across the two WSI scanners. A color chart slide (e.g., the color chart slide of FIG. 17) can be utilized to calibrate scanner variation. A calibrator slide (e.g., the calibrator slides described herein, including the calibrator slide of FIG. 16) includes peptide-coated microbeads with different concentration is used to calibrate staining and scanner variation. Further details and approaches for color calibration including these techniques are described herein, in the context of the functionality implemented by the data processing system 105 of FIG. 1.

In this non-limiting example experiment, two scanners (e.g., the scanner devices 125) can be used to evaluate both staining and scanning variation. If more than two scanners are utilized, these techniques can be used to calibrate both variations. Results of automated HER2 evaluation between the combination of the calibrator slide and the color chart slide and the use of the calibrator alone in connection with this example experiment are described in further detail herein. The non-limiting example experiment can utilize the color chart slide (e.g., of FIG. 17), calibrator slide (e.g., of FIG. 16), and HER2 IHC stained slides (e.g., as described herein) of breast cancer excision cases with known scores for evaluation.

In this non-limiting example experiment, four HER2 0-3+ breast cancer cases were selected for evaluation, and formalin-fixed paraffin-embedded breast cancers were sectioned at 4 μm and 6 serial section slides were prepared for each case. In this non-limiting example experiment, two types of calibration slides are used: for staining variation, a calibrator slide, and for scanning variation, a color chart slide. The calibrator for HER2 (e.g., shown in FIG. 16) can include two different microbeads. Larger microbeads are test microbeads coated with 10 different levels of peptide concentration and stained with DAB by IHC staining. Smaller microbeads are colored brown as showing standard DAB color.

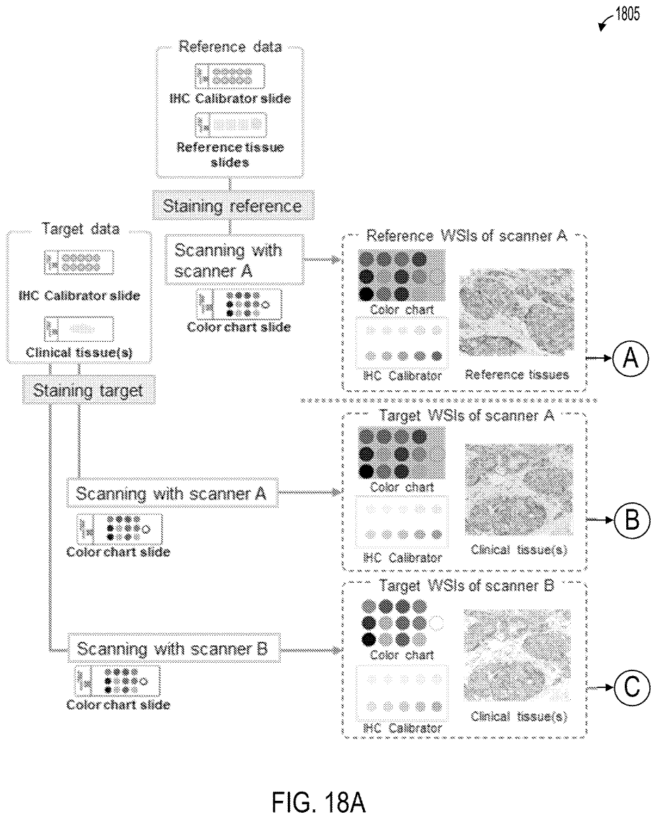

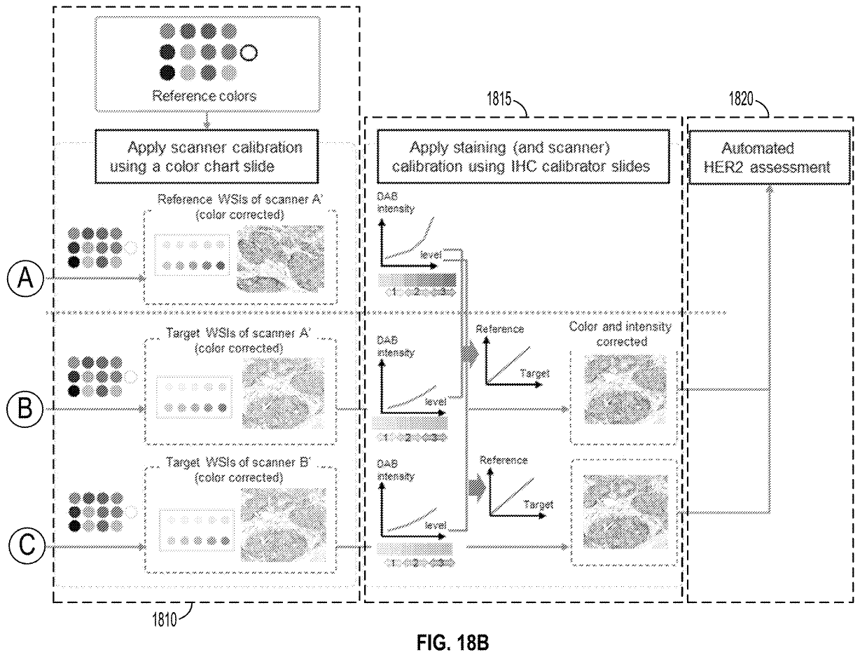

The color chart slide (e.g., shown in FIG. 17) can be utilized calibrate, standardize, and trace color settings for imaging analysis. The color chart slide can include a glass slide embedded with twelve color patches and a background area. Five slides comprising HER2 0-3+ slides and the calibrator slide were utilized as a dataset. To obtain the slides that reflect the daily variation in staining, the datasets were stained at different dates continuously using PATHWAY anti-HER2/neu Antibody. Six IHC stained datasets were prepared. The stained slides and the color chart slide were digitized by two WSI scanners (e.g., the scanner devices 125 of FIG. 1), at 0.23 μm per pixel resolution (40× equivalent) and at 0.18 μm/pixel resolution (20× equivalent). Exported bitmap image data were used for evaluation. The evaluation process for these example slides for this non-limiting example experiment is shown in FIGS. 18A and 18B.

Referring to FIGS. 18A and 18B in the context of the components described in connection with FIG. 1, illustrated is an example flow diagram of a process for color and intensity standardization, including scanner calibration and staining calibration, which may be implemented by the system of FIG. 1, in accordance with one or more implementations. FIG. 18A shows step 1805 of the flow diagram, in which the scanner devices 125 (shown here individually as “scanner A” and “scanner B”) can be utilized to generate reference WSIs of scanner A, and respective target WSIs of scanner A and scanner B. This process can be utilized to address both sub-issues of staining process dependence and scanner dependence.

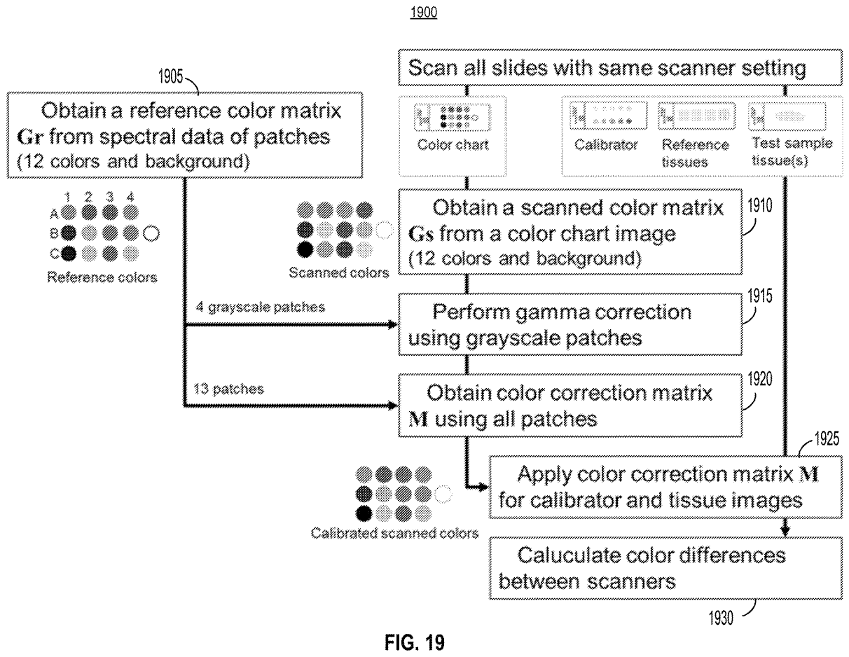

In step 1810, color variations depending on the WSI scanner are calibrated using the color chart slide. Then, in step 1815, color intensity variations depending on the staining process are calibrated using the calibrator slide, using the various techniques described herein (e.g., in connection with FIG. 19, etc.). Finally, at step 1820, standardized images are evaluated with automated image analysis software for HER2, as described herein. Even if the color chart slide is not used, the calibrator slide can diminish scanner-caused variations.

Referring to FIG. 19 in the context of the components described in connection with FIG. 1, illustrated is an example flow diagram of a process 1900 that may be implemented by the system 100 of FIG. 1 for scanner calibration, in accordance with one or more implementations. In the process 1900, each scanner image can be color standardized to approach to the reference colors of the color chart slide. Additionally, the stained slides and the color chart slide can be scanned with the same settings (e.g., by the scanning devices 125 of FIG. 1).

In step 1905, reference colors are acquired by using a supplied spreadsheet containing spectral transmission data for each patch on the slide. In step 1910, scanned colors are obtained from the image. In step 1915, before calculating a color correction matrix, ascertain whether the gamma of the scanned image is linear. If the color correction matrix includes gamma correction (e.g., the gamma curve is not linear), gamma correction can be performed first. In step 1920, the color correction matrix is derived from the reference and scanned colors. In step 1925, the color correction matrix is applied to other images to obtain color-standardized images. Steps 1910 through 1925 can be applied to each scanner image. To evaluate the effect of color calibration, color differences between images from different scanners are calculated in step 1930.

In step 1905 of the process 1900, the data processing system 105 or one or more of the components thereof (e.g., the intensity profile determiner 135) can calculate reference colors of the color chart slide from their representative spectral transmittance samples. Each color patch has spectral transmittance T(λ) in 5 nm increments, from 340 to 830 nm, for example. The CIEXYZ color space represents colors by the tristimulus values X, Y, and Z of object colors by reflection. Tristimulus values for each patch are calculated using the following equations:

$$X = k {\int }_{340}^{830} T ( λ ) S ( λ ) \overline{x} ( λ ) d λ$$

$$Y = k {\int }_{340}^{830} T ( λ ) S ( λ ) \overline{y} ( λ ) d λ$$

$$Z = k {\int }_{340}^{830} T ( λ ) S ( λ ) \overline{z} ( λ ) d λ$$

$$k = 1 / {\int }_{340}^{830} S ( λ ) \overline{y} ( λ ) d λ$$

In the above equations, S(λ) is the value of spectral distributions of illuminant source at the wavelength λ, x̅(λ), y̅(λ), and z̅(λ) are the CIE color matching function for the Standard Observer, respectively. The factor k normalizes the tristimulus value so that Y will have a value of 1.0 for the perfect white diffuser. Obtained tristimulus values T=(X Y Z)^{t} are converted to the linear RGB values R=(R G B)^{t} by multiplying with coefficients of 3×3 XYZ to RGB conversion matrix C, as shown in the equation below.

$$T = CR$$

In this non-limiting example experiment, illuminant D65 is used as the light source S(λ). A 3×13 matrix Gr which contains the reference RGB values of 12 color patches and background was obtained, where FIG. 17 shows the calculated reference colors.

In step 1910 in the process 1900, the data processing system 105 or one or more of the components thereof (e.g., the intensity profile determiner 135, etc.) can obtain the scanned colors of the color chart image. RGB values at each pixel are normalized by the data processing system 105 by dividing with incident light as shown in the equation below.

$$R = I ∅ {I}_{0}$$

In the equation above, R=(R_{R} R_{G} R_{B})^{t} is the RGB vector at each pixel in the image, I₀=(I_{0R} I_{0G} I_{0B})^{t} is the RGB intensity vector of the incident light, I=(I_{R} I_{G} I_{B})^{t} is the RGB intensity vector of the light passed through the patches, and Ø denotes Hadamard division operator, respectively. By normalizing with I₀ selected from a glass area without a specimen and taking a logarithm, optical density for each wavelength is linear with the stain intensity. The average value of the central area of each patch is calculated, then a 3×13 matrix Gs which contains the scanned normalized RGB values is obtained.

In step 1915 of the process 1900, the data processing system 105 or one or more of the components thereof can utilize grayscale colors (A1, B1, C1, and background in FIG. 17) for gamma correction. Gamma, represented by γ, can be described as the relationship between an input x and an output y, as shown in the equation below.

$$y = {x}^{γ}$$

In this non-limiting example experiment, the relationship between the reference and scanned colors can be derived from the tristimulus value Y of grayscale colors. If the scanned image is linear (e.g., gamma of 1.0), scanned values equal the reference values and produce a straight line. If the scanned image is non-linear, the data processing system 105 can correct all pixel values with the obtained gamma.

In step 1920 of the process 1900, the data processing system 105 or one or more of the components thereof can derive the color correction matrix, M, using the reference and scanned color matrices, Gr and Gs, based on the equations below.

$${G}_{r} = {G}_{s} M$$

M may be determined by:

$$M = {G}_{s}^{- 1} {G}_{r}$$

In the equations above, ·⁻¹ denotes the inverse of the matrix. When Mis a square matrix, a solution can be obtained by computing the inverse matrix. Otherwise, a solution may be obtained by computing the Moore-Penrose pseudoinverse, and the above equation becomes as follows.

$$M = {G}_{s}^{+} {G}_{r}$$

In some implementations, another approach to finding M includes using white-point preserved least-square (WPPLS). This approach finds the M that minimizes the overall residual square error and at the same time preserves the background white, as shown in the following equation.

$${G}_{r} = {G}_{s} M and uM = u$$

In the above equation, u is a row vector of the background, equal to (1,1,1)^{t}. Linear color correction is utilized to find the 3×3 color correction matrix M. In some implementations, higher-order polynomial (nonlinear) transforms can be used. In this non-limiting example experiment, WPPLS and the polynomial transformation were combined to obtain M. M is obtained for each scanner (e.g., each scanner device 125). In step 1925, the obtained M is applied to other images then color of image is standardized.

In step 1930 of the process 1900, to express the color difference between images, the data processing system 105 can correlate the CIELAB color space. L*, which is correlated with brightness, a* and b*, with the color opponent dimensions. The XYZ to L*a*b* conversion is defined in the following equations.

$${L}^{*} = 116 f ( \frac{Y}{{Y}_{0}} ) - 16$$

$${a}^{*} = 500 [ f ( \frac{X}{{X}_{0}} ) - f ( \frac{Y}{{Y}_{0}} ) ]$$

$${b}^{*} = 200 [ f ( \frac{Y}{{Y}_{0}} ) - f ( \frac{Z}{{Z}_{0}} ) ]$$

$$f ( x ) = \{ \begin{matrix}{x}^{1 / 3} & x > 0.00886 \\ 7.787 x + \frac{16}{116} & x \leq 0.00886\end{matrix}$$

In the above equations, X₀, Y₀, and Z₀ correspond to the tristimulus values of the reference white point. The color difference dE* between two colors (L₁*, a₁*, b₁*) and (L₂*, a₂*, b₂*) is proportional to the Euclidian distance, which is calculated using the equation below.

$${dE}^{*} = \sqrt{{( {L}_{1}^{*} - {L}_{2}^{*} )}^{2} + {( {a}_{1}^{*} - {a}_{2}^{*} )}^{2} + {( {b}_{1}^{*} - {b}_{2}^{*} )}^{2}}$$

When the calculated dE* is between 1.0 and 2.0, only an experienced observer can notice the perceptual difference, dE is below 1.0, an observer cannot notice the difference.

FIGS. 20A and 20B show an example flow diagram of a process 2000 for staining calibration, which may be implemented by the system of FIG. 1, in accordance with one or more implementations. The use of automated image analysis software for HER2 test is assumed. The process 2000 includes a preparation stage, shown in FIG. 20A and an evaluation stage, shown in FIG. 20B. In this non-limiting example experiment, the calibrator slide is stained in each step to provide comparable staining references.

In the preparation stage of FIG. 20A, the calibrator slide and score known breast cancer cases are prepared as a reference dataset in step 2002. The reference dataset is digitalized with a single scanner (e.g., one of the scanner devices 125) that is regarded as a reference scanner. In some implementations, if the automated image analysis software is intended to use a specific scanner, that scanner becomes the reference. In the evaluation stage shown in FIG. 20B, another calibrator slide is stained and scanned together with the clinical tissue slides to be assessed to generate the target dataset 2012. If the same scanner is used for the digitization of the reference and target dataset, the proposed process 2000 implies staining correction. If the scanners are different (e.g., difference scanner devices 125), the process 2000 has the effect of both scanner correction and staining correction. When combining scanner calibration, the color chart slide can be scanned together at each stage.

For standardization of HER2 IHC stained images, the color intensity of DAB can be calibrated. A color un-mixing method, which is based on an optical model, can be used to separate a color image of multi-stained specimens into the components of stain intensities representing the single stains. The light extinction follows Lambert-Beer's law. The optical density (OD) measured from the image is linearly related to the stain amount, as shown in the equation below.

$$O = - {log}_{10} ( I ⌀ {I}_{0} ) = VC$$

In the equation above, O=(O_{R} O_{G} O_{B})^{t} is the RGB vector of the OD at each pixel in the image, V is the stain matrix whose columns are the unit vectors of RGB absorption coefficients of staining colors, and C is the vector of which each element represents the stain intensity, respectively. In the vector-matrix notation here, the spatial coordinates are omitted for simplicity. Once Vis determined, C can be determined by the data processing system 105 (or one or more of the components thereof) using the following equation.

$$C = {V}^{+} O$$

When the specimen is a tissue stained with DAB and hematoxylin, the OD is expressed by the model below:

$${O}_{tissue} = {V}_{DAB} {C}_{DAB} + {V}_{H} {C}_{H}$$

In the equation above, V_{DAB} is the stain matrix of DAB, and V_{H} is the stain matrix of hematoxylin. These matrices are needed for color un-mixing to derive the stain intensities C_{DAB} and C_{H}.

Referring to the preparation stage of the process 2000 shown in FIG. 20A, the data processing system 105 can obtain the staining color and intensity of DAB V_{DAB,R} and C_{DAB,R} from the microbeads of each level in the calibrator image. To do so, in step 2005, the data processing system 105 can obtain the color of hematoxylin VHR is obtained from the 0-scored images since the tissue is stained with hematoxylin only, and subsequently measure the stain intensity from the calibration image using an automated image analysis technique in step 2010. As shown, the reference dataset 2002, which includes images of reference tissues as described herein, is utilized.

Referring to the evaluation stage of the process 2000 shown in FIG. 20B, the data processing system 105 can obtain the staining color and intensity for target V_{DAB,T} and C_{DAB,T} are obtained the images of the calibrator slide produced as described herein. The data processing system 105 can calculate the intensity correction function ƒ(C) to map the stain intensity of the target dataset to that of the reference dataset. The data processing system 105 can obtain the linear function ƒ(C) using regression analysis, minimizing |C_{DAB,T}−ƒ(C_{DAB,R})|².

As shown, the process 2000 includes selecting between at least two methods of automated assessment in step 2015. The use of multiple implementations allows the data processing system 105 (or an operator of the data processing system 105, via user input) to select a suitable correction method depending on the automated image analysis software. For example, if the image analysis software is threshold adjustable, the approach in step 2020 can be utilized. Otherwise, the step 2025 can be selected.

In step 2020 of the process 2000, the data processing system 105 can adjust the thresholds for classifying DAB membranous intensities by using ƒ(C). In step 2025 of the process 2000, the data processing system 105 can correct the color and intensity of tissue images to match the reference image using ƒ(C) and the stain matrices. In this non-limiting example experiment, and as described herein above, two scanners (scanner A and scanner B, each of which may be one of the scanner devices 125) are utilized, where scanner A is considered the reference scanner in the staining calibration step.

Referring back to FIG. 1, and further detailing the non-limiting example experiment described above, scanner calibration was performed on all scanned images using the techniques described herein. To evaluate the perceptual color difference between two images pixel-wise, dE* maps were created. The dE* map shows dE* between two images calculated for each corresponding pixel as pixel values. Since exported images of two scanners have different spatial dimensions and pixel resolutions, image registration can be performed for experimental evaluation. In this non-limiting example experiment, AKAZE (Accelerated-KAZE), which detects and matches key points on two images, was utilized for image registration. To determine the order of polynomial transformation, dE* was calculated for different combinations of orders of two scanners. The combination with the smallest color difference was selected.

Additionally, in this non-limiting example experiment, a simulation experiment was conducted to see if including brown would reduce the color difference in IHC staining. The color correction matrix was derived using 3×14 matrices Gr′ and Gs′, which are originally 3×13 matrices Gr and Gs, with brown added. The brown was obtained from the IHC calibrator images. Reference color was obtained from the larger microbeads in scanner A, images that were color calibrated using Gr and Gs. Scanned color was obtained from the smaller microbeads of each scanner's original images. To check whether the intensity of brown color affects color calibration, two patterns of color intensity were prepared: bright brown and dark brown. Bright brown corresponds to the intensity of the larger microbeads on level 10. Dark brown corresponds to the intensity of the smaller microbeads.

In this non-limiting example experiment, further analysis was performed using automated HER2 assessments. Automated HER2 assessments were performed on the images before and after calibration. Accuracy was assessed after performing different combinations of scanner and staining calibration, as shown in Table 1 below. When performing staining calibration, one of the 6 datasets was regarded as a reference dataset, and the remaining 5 datasets were regarded as target datasets for HER2 test.

TABLE 1

| | | Staining | Staining |
|---|---|---|---|
| | | Calibration: | Calibration: |
| | Scanner | Thresholds | Images |
| Combination | Calibration | (Step 2020) | (Step 2020) |
| A | No | No | No |
| B | No | Yes | No |
| C | No | No | Yes |
| D | Yes | No | No |
| E | Yes | Yes | No |
| F | Yes | No | Yes |

The data processing system 105 can execute automated image analysis software for HER2 score classification. A non-limiting example of such software includes QuPath. The software executed by the data processing system 105 may be semi-automatic, and can enable setting color vectors for color un-mixing and thresholds for classification. The data processing system 105 can calculate the histoscore (H-score), which is one of the evaluation indexes in HER2 assessment, by solving the following equation.

$$H - score = P \times I$$

In the above equation, I is immunoscore (0, 1, 2, or 3) classified according to the thresholds and P is the percentage of immunoreactive tumor cells, respectively. The data processing system 105 can calculate the standard deviation (SD) of the H-score. The SD was utilized to compare the combinations of calibration in this non-limiting example experiment. A two-way analysis of variance (ANOVA) was performed to analyze the effect of scanner calibration and staining calibration on automated HER2 assessment. Tukey's honest significant difference (HSD) test was also utilized as a post hoc analysis.

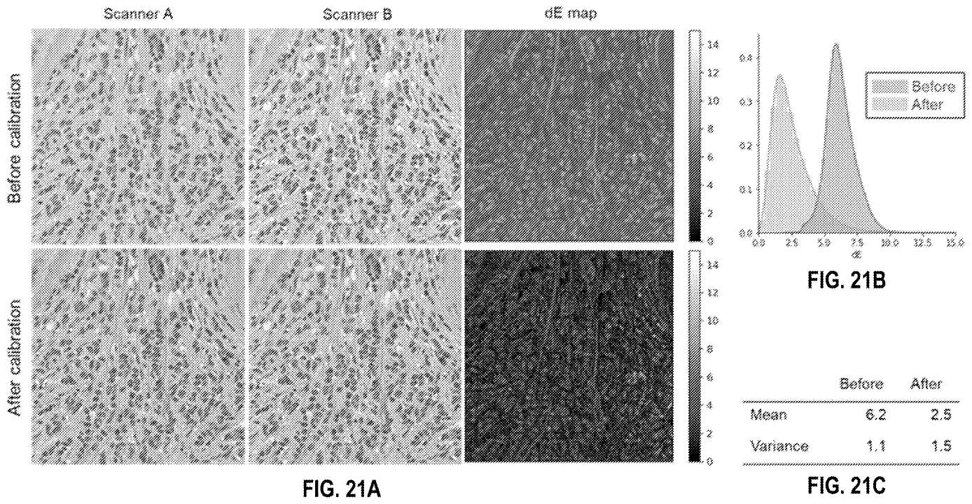

FIGS. 21A, 21B, and 21C; 22A, 22B, and 22C; and 23A, 23B, and 23C show example results of scanner calibration with the color chart slide based on the techniques described herein. The top panels of FIGS. 21A, 22A, and 23A show uncalibrated images and the lower panels show the color-calibrated images. By performing calibration using the techniques described herein, the mean values of color difference were reduced in H&E stained tissue images (from 6.2 to 2.5) and IHC stained tissue images (4.1 to 1.6), and IHC stained calibrator images (4.6 to 4.1).

FIGS. 21A, 21B, and 21C show example slide images, plots, and mean and variance values, respectively, for example stained breast cancer tissue images, in accordance with one or more implementations. FIG. 21A shows a comparison of images before and after color calibration and dE* maps based on the techniques described herein. The dE* maps show the color differences between two scanners calculated for each corresponding pixel as pixel values. FIG. 21B shows example histograms of the dE* maps before and after calibration. FIG. 21C shows the mean and variance values of the dE* before and after calibration. Images are shown for both scanner A and scanner B used in the non-limiting example experiment.

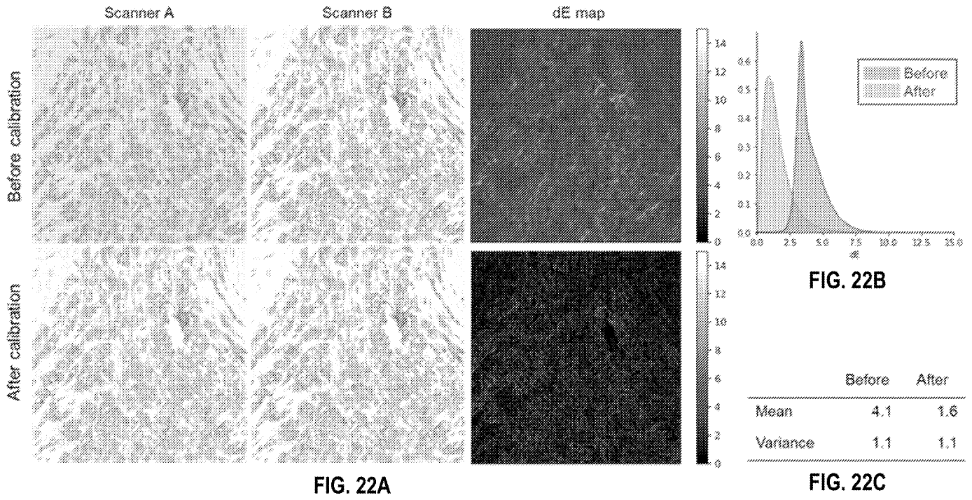

FIGS. 22A, 22B, and 22C show example slide images, plots, and mean and variance values, respectively, for example HER IHC stained breast cancer tissue images, in accordance with one or more implementations. FIG. 22A shows a comparison of the HER IHC stained breast cancer tissue images before and after color calibration, and their corresponding dE* maps. FIG. 22B shows example histograms of the dE* maps before and after calibration. FIG. 22C shows the mean and variance values of the dE* before and after calibration. Images are shown for both scanner A and scanner B used in the non-limiting example experiment.

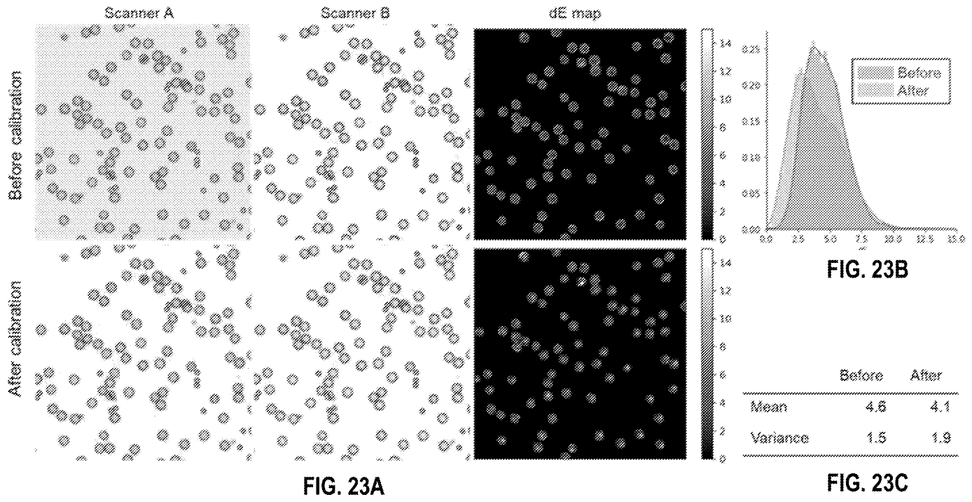

FIGS. 23A, 23B, and 23C show example slide images, plots, and mean and variance values, respectively, for example IHC calibrator images, in accordance with one or more implementations. FIG. 23A shows a comparison of the IHC calibrator images before and after color calibration, and their corresponding dE* maps. FIG. 23B shows example histograms of the dE* maps before and after calibration. FIG. 23C shows the mean and variance values of the dE* before and after calibration. Images are shown for both scanner A and scanner B used in the non-limiting example experiment.

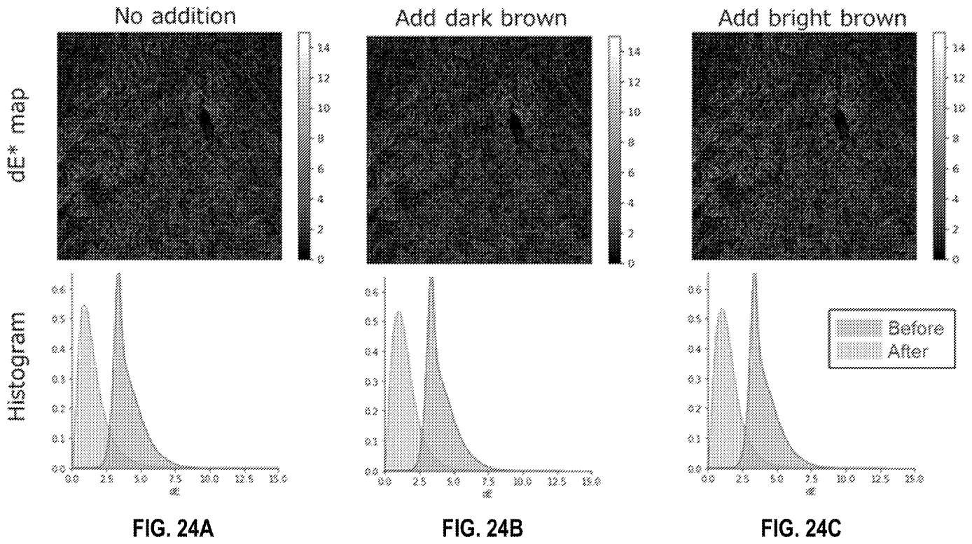

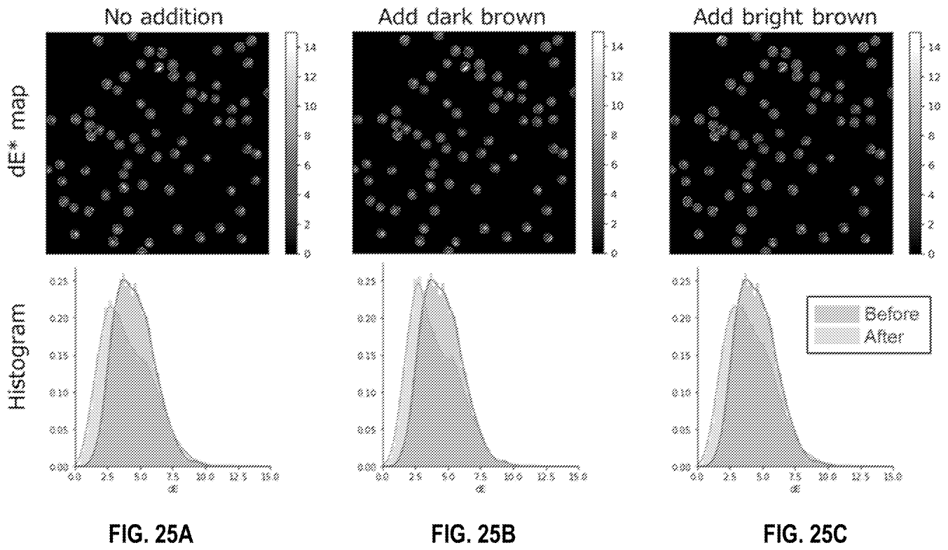

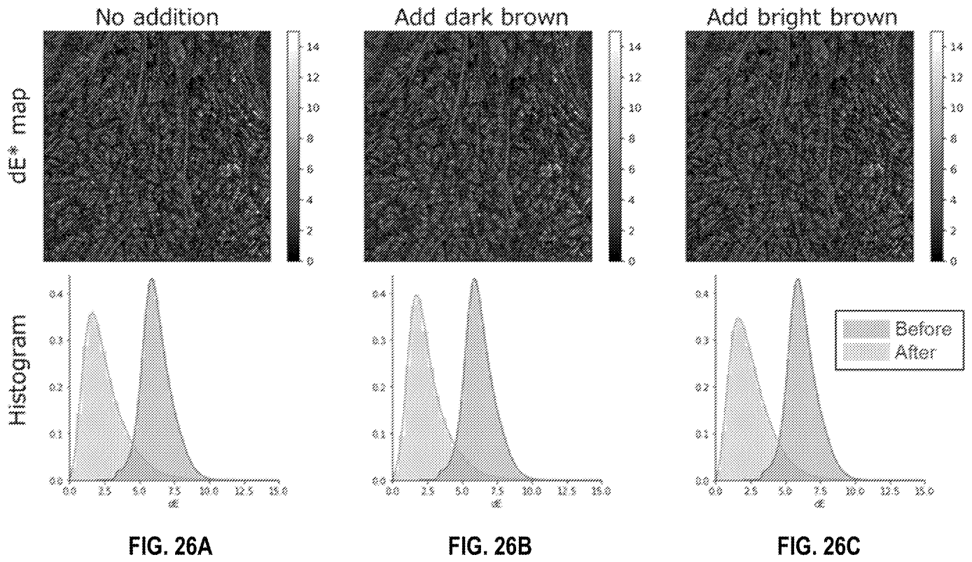

FIGS. 24A, 24B, and 24C show comparisons of dE* maps with and without brown in HER2 IHC stained breast cancer tissue images. FIGS. 25A, 25B, and 25C show comparisons of dE* maps with and without brown in IHC stained calibrator images. FIGS. 26A, 26B, and 26C show comparisons of dE* maps with and without brown in H&E stained breast cancer tissue images. FIGS. 24A-26C show comparison results of color calibration with and without brown color. Table 2 below shows the percentage of dE* larger than 6 and the mean value of dE*.

In these non-limiting example results, when dark brown was added, the percentage of large color difference became smaller in IHC stained tissue (from 0.5 to 0.2) and calibrator images (16.9 to 14.8). In the H&E stained images, dE* became smaller by adding brown color. Table 3 below shows parameters obtained by analyzing the relationship of DAB intensity between scanners A and B. The parameter would ideally be 1.0 since it is the relationship for the DAB intensities of same calibrator slide. Scanner calibration brought it closer to 1.0 even without the addition of brown.

TABLE 2
Comparison of color difference with and without brown

| | IHC stained tissue | | | | IHC stained calibrator | | | | H&E stained tissue | | | |
|---|---|---|---|---|---|---|---|---|---|---|---|---|
| | | After | | | | After | | | | After | | |
| | | No | Dark | Bright | | No | Dark | Bright | | No | brown | Bright |
| | Before | add. | brown | brown | Before | add. | brown | brown | Before | add. | Dark | brown |
| Percentage | 6.1 | 0.5 | 0.2 | 0.6 | 17.4 | 16.9 | 14.8 | 14.9 | 55.2 | 3.1 | 2.8 | 2.6 |
| of dE* larger | | | | | | | | | | | | |
| than 6 (%) | | | | | | | | | | | | |
| Mean of dE* | 4.1 | 1.6 | 1.5 | 1.6 | 4.6 | 4.1 | 4.0 | 4.1 | 6.2 | 2.5 | 2.5 | 2.5 |

TABLE 3
Parameter obtained by analyzing the relationship
of DAB intensity between two scanners

| | | After | | |
|---|---|---|---|---|
| | | No | Dark | Bright |
| | Before | addition | brown | brown |
| Parameter | 0.90 | 1.05 | 1.02 | 1.05 |

Furthering the non-limiting example results of the aforementioned experiment, Table 4 below shows the result of the automated HER2 assessments described herein. Without staining calibration (Methods A and D of Table 1 above), there were some misclassified slides. However, with both scanner and staining calibration (Methods B, C, E, and F of Table 1 above), results showed concordance of 100% with the pathologist's assessment. Also, SD of the H-score became smaller with both calibrations.

Table 5 below shows the result of a two-way ANOVA, and Table 6 below shows the result of a Tukey HSD test, as described herein. The two-way ANOVA revealed that there was not a statistically significant interaction between scanner and staining calibrations. Simple main effects analysis showed that scanner calibration had a statistically significant in 1+ case, F (1, 54)=22.30, P=0.000, and in 3+ case, F (1, 54)=14.09, P=0.000. Simple main effects analysis showed that staining calibration had a statistically significant in 3+ case, F (2, 54)=3.63, P=0.033. Post hoc analyses using Tukey's HSD test for significance indicated showed that the method was significant in 2+ between A and E (P=0.002), A and F (P=0.002), in 3+ case between B and D (P=0.047), C and D (P=0.001), C and E (P=0.035).

TABLE 4
Results of HER2 score classification

| | | | | | | | Standard deviation of | | |
|---|---|---|---|---|---|---|---|---|---|
| | Scanner | Staining calibration | | Concordance (%) | | | H-score | | |
| Methods | calibration | Method 1 | Method 2 | 1+ | 2+ | 3+ | 1+ | 2+ | 3+ |
| A | No | No | No | 90 | 80 | 100 | 10.5 | 10.3 | 8.3 |
| B | No | Yes | No | 100 | 100 | 100 | 6.7 | 7.0 | 5.6 |
| C | No | No | Yes | 100 | 100 | 100 | 7.9 | 7.6 | 6.1 |
| D | Yes | No | No | 70 | 90 | 100 | 15.9 | 12.6 | 10.1 |
| E | Yes | Yes | No | 100 | 100 | 100 | 11.9 | 10.8 | 7.8 |
| F | Yes | No | Yes | 100 | 100 | 100 | 11.0 | 8.2 | 7.4 |

TABLE 5
Results of a two-analysis of variance

| HER2 | | | |
|---|---|---|---|
| score | | F | P |
| 1+ | Scanner | 22.30 *** | .000 |
| | Stain | 2.46 | .095 |
| | Interaction | 0.01 | .991 |
| 2+ | Scanner | 1.21 | .277 |
| | Stain | 1.73 | .187 |
| | Interaction | 0.07 | .934 |
| 3+ | Scanner | 14.09 *** | .000 |
| | Stain | 3.63 * | .033 |
| | Interaction | 0.27 | .762 |

TABLE 6
Results of post hos analysis;

| | | | | | 95% confidence | |
|---|---|---|---|---|---|---|
| HER2 | | | | | interval | |
| score | group 1 | group 2 | Mean diff. | p | Lower | Upper |
| 1+ | A | E | −20.85 ** | .002 | −36.06 | −5.63 |
| | A | F | −22.06 ** | .002 | −36.27 | −5.84 |
| 3+ | B | D | 10.79 * | .047 | 0.08 | 21.51 |
| | C | D | 15.30 ** | .001 | 4.59 | 26.02 |
| | C | E | 11.24 * | .035 | 0.53 | 21.95 |

* P < 0.05
** P < 0.01
***P < 0.001

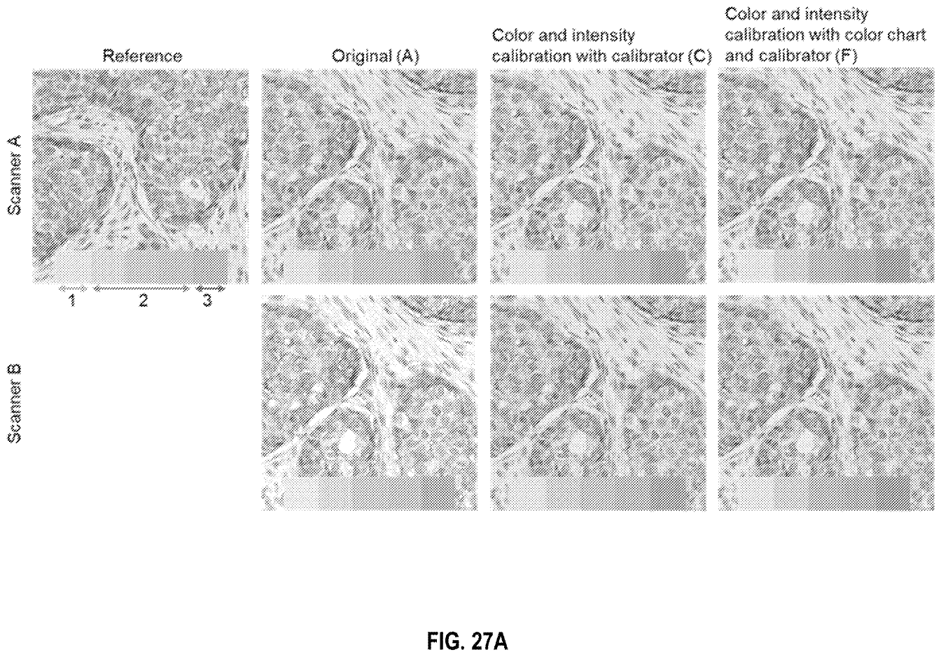

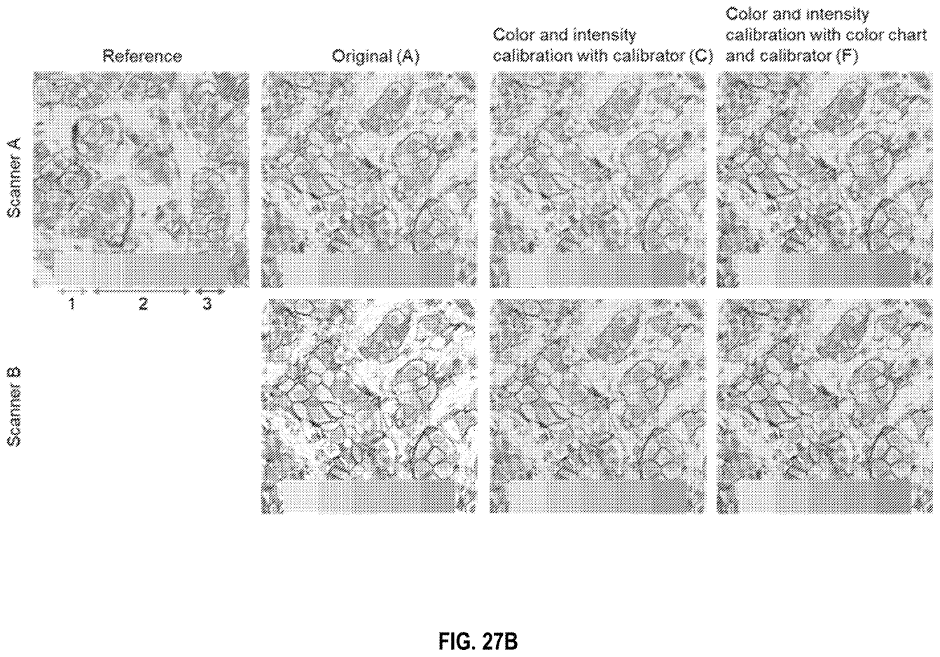

Referring to FIGS. 27A and 27B, depicted are HER2 IHC breast cancer images scanned using a 40× objective lens with intensity color bar. FIGS. 27A and 27B compare original images and color and intensity calibrated images captured from scanner A and scanner B of the above-descripted non-limiting example experiment, with FIG. 27A depicting a 2+ case and FIG. 27B depicting a 3+ case. FIGS. 27A and 27B show examples of color and intensity calibrates images (A, C, and F in Table 4 above). The intensity color bars shown below the tissue images were generated from the color intensity of calibrator images. The color of the target images was corrected to approach the reference images.

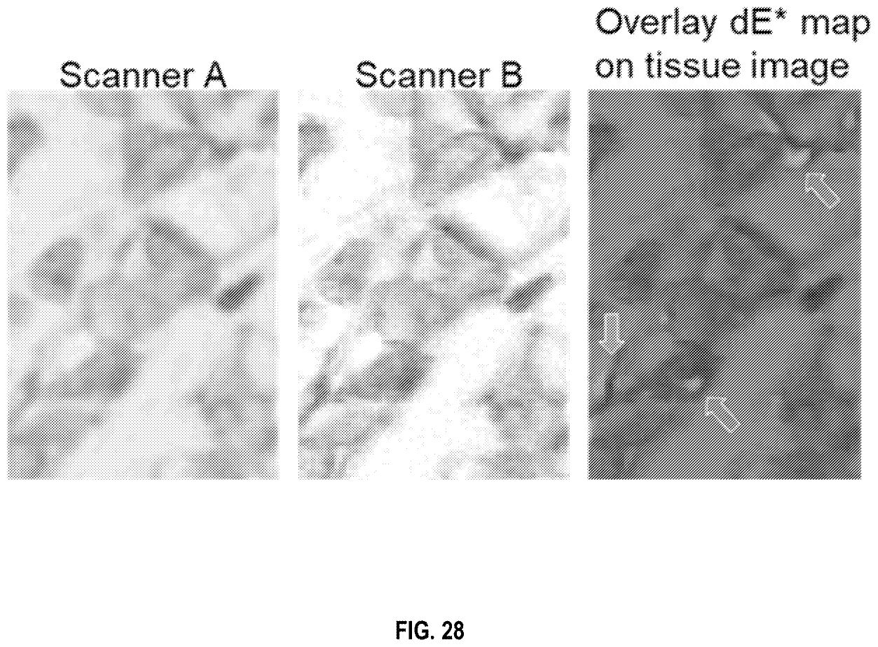

Referring to FIG. 28, depicted is a comparison of tissue images captured from scanner A and scanner B of the above-described non-limiting example experiment, and a corresponding dE* map. The dE* map is overlaid on the tissue image with a magenta color. The techniques described herein, which may utilize the color chart slide of FIG. 17, allows for standardizing color of WSI, which varies depending on scanning devices (e.g., the scanner devices 125). The color differences between scanners have been reduced in both H&E and IHC staining. Especially for IHC staining, dE was reduced to less than 2.0, it is said that only experienced observer can notice the difference.

The dE* in HER2 IHC stained tissue tends to be larger in areas where DAB is stained dark (red arrows in FIG. 28). Adding dark brown when determining the color correction matrix reduced the dE* in dark areas. From the results of calibrator slide shown in FIGS. 25A, 25B and 25C, the effect is noted when a darker brown was added than when corresponding intensity was added. From the results of Table 3, the effect is sufficient even without the brown. There were some color differences around the membrane (green arrow in FIG. 28), which may be a result of differences in the spatial frequency of the scanners. This is because when color is standardized through scanner calibration, other factors may cause color differences.

In this non-limiting example experiment, the accuracy after performing different combinations of scanner and staining calibration were assess. The results of this non-limiting example experiment, as shown in part in Table 4, indicate that misclassification was eliminated by performing both calibrations. Additionally, the SD of the H-score became smaller by applying both calibrations. The SD tends to be smaller for Methods A-C of Table 1, which do not use the color chart slide, when compared to Methods D-F of Table 1. A difference was observed between methods C and E of Table 1, in the 3+ tissue sample. This result means that Method C may have improved performance compared to method E. Using automated HER2 assessment, it is possible to evaluate with high accuracy without using the color chart slide.

The techniques described herein using the calibrator slide are designed to use reference scanner. By utilizing the techniques described herein, images scanned by other scanners can be analysed using information from the reference scanners. The techniques described herein can be introduced into clinical workflows, since the only additional processes in these techniques to perform the same staining protocol to a calibrator slide. When performing scanner calibration using the color chart slide, gamma correction is performed before color correction. However, if only the calibrator slide is used, gamma is not corrected. In some implementations, a scanner with linear characteristics can be used as a reference.

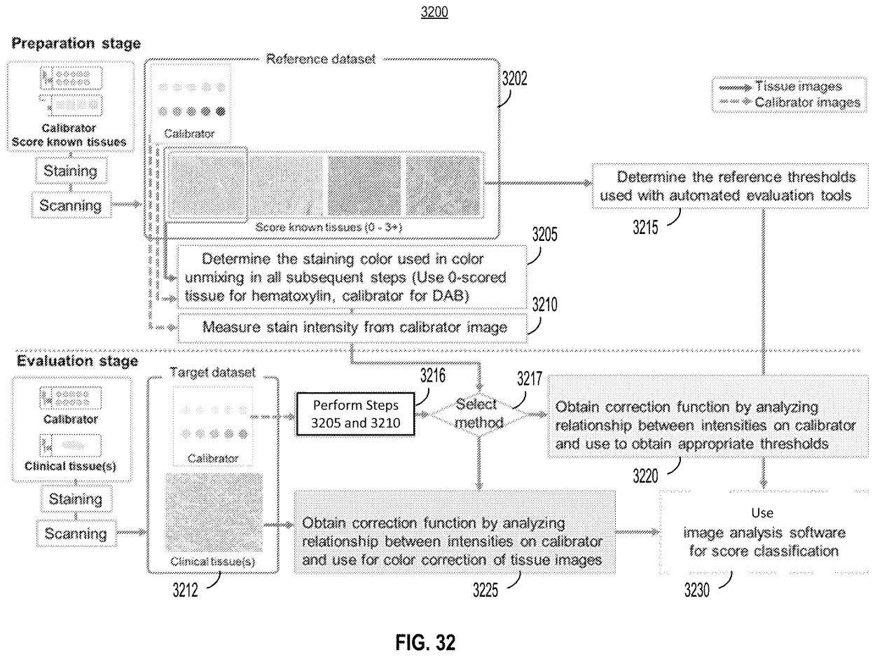

An additional non-limiting example experiment is described herein below, which may be implemented using the techniques described in connection with the above-example experiment. These techniques are described in connection with steps of the process 3200 of FIG. 32, which is described herein below and may be similar to the steps of the process 2000 described in connection with FIGS. 20A and 20B.

In the additional non-limiting example experiment, a calibrator slide (e.g., any of the example calibrator slides described herein) and tissue of breast cancer slides were stained together at different dates continuously and scanned with a WSI scanner (e.g., a scanner device 125 of FIG. 1). The additional non-limiting example experiment, sixty images were used to evaluate the techniques described herein. The results show that, in implementations that do not utilize the techniques described herein, HER2 status of seven slides out of 60 slides were misclassified using an automated image analysis software.

However, using the techniques described herein, the misclassification was almost eliminated (0/60 slide for one implementation, 1/60 slide in another implementation). Further, of the additional non-limiting example experiment showed that the present techniques can could significantly reduce the variability of the score of HER2 intensity. An analysis of variance showed that the effect of the method was significant comparing to the evaluation without calibrator, in 2+ cases, F (2, 40)=11.79, P=0 and in 3+ cases, F (2, 40)=5.14, P=0.010. Therefore, the proposed techniques enable calibration of DAB intensity and evaluation of HER2 status with more consistency, which is an improvement over conventional techniques. The automated assessment of other IHC tests (membrane or cytoplasmic stain which assess intensity of stain) is also possible using the present techniques.

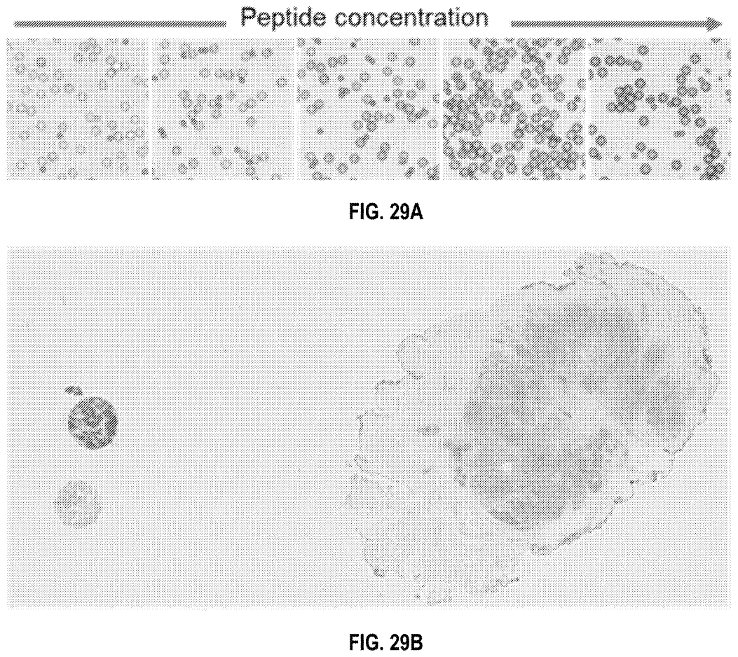

Referring to FIGS. 29A and 29B, depicted are HER2 IHC stained samples using a 40× objective lens. FIG. 29A shows microbeads on the IHC calibrator slide. FIG. 29B shows negative and positive controls for IHC test (left) on the same slide of HER2-positive breast cancer sample. The calibrator slide for HER2 depicted in FIG. 29A is made of microbeads coated with different amounts of peptide concentration, as described herein. The calibrator slide can be used to ascertain whether each laboratory's immunostain is configured correctly. Negative and positive controls can be run with every assay to help ensure staining quality in IHC. Including the controls on the same slide as the tissue sample can assist pathologists to visually evaluate the DAB intensity, but positive controls often have even higher intensity than clinical positive samples, as shown in FIG. 29B. In some cases, controls derived from tissue samples are of unknown analyte concentration and will be variable as samples are changed. However, the IHC stained calibrator slide has microbeads with an intensity range that is appropriate for the tissue samples, with various intensities depending on concentration level. The views of the calibrator slide shown FIGS. 29A and 29B is more reproducible since concentration for each level is defined.

In this additional non-limiting example experiment, the calibrator slide is used to handle the color intensity variation of WSI due to the staining process. As described herein, the processes performed on the calibrator slide can be performed together with tissue sample slides, from staining to WSI scanning. The calibrator slide may also be used in microscopy. The techniques described herein utilize the WSI of the calibrator slide and enable the automated calibration of DAB color intensity in the tissue image. The DAB color intensities of microbeads in the calibrator image correlate with those of the tissue sample images. The DAB color is obtained from the color of microbeads, and the intensity characteristics are derived from the densities of the different levels of microbeads. The present techniques may be utilized with an automated image analysis software for HER2 test, as described herein.

As described herein in connection with FIG. 32, two experimental implementations may be selected from depending on the automated image analysis software. One implementation (e.g., step 3220 of the process 3200 in FIG. 32) includes setting the thresholds for classifying HER2 status based on the intensity of the microbeads of every level, and another implementation (e.g., step 3225 of the process 3200 in FIG. 32) includes performing color correction of tissue images. Further details of each of these steps are described in further detail herein below. This additional non-limiting example experiment evaluates the performance of the present techniques when correcting for the variation created during to the staining process, and utilizes the calibrator slide and HER2 IHC stained slides of breast cancer excision cases with known scores.

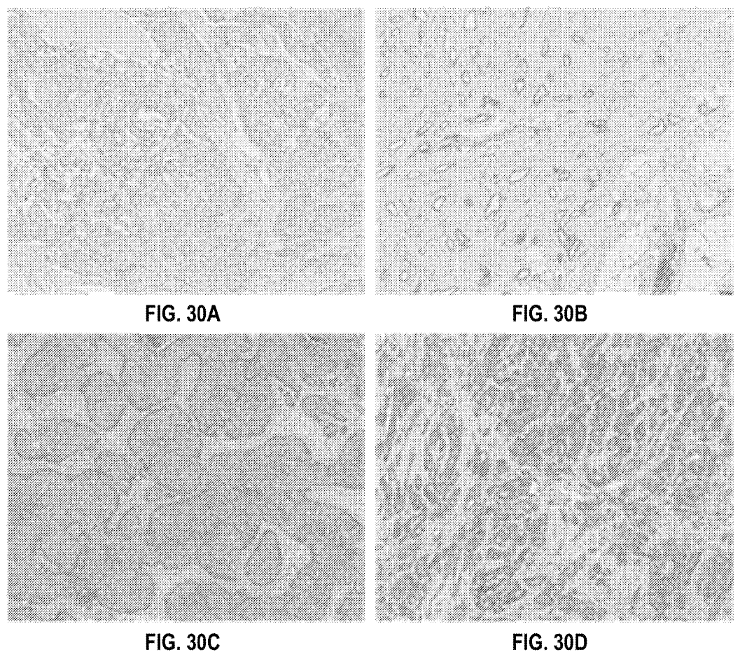

Referring to FIGS. 30A, 30B, 30C, and 30D, depicted HER2 IHC stained breast cancer images scanned at 0.23 μm/pixel resolution (40× equivalent) for a 0 case, a 1+ case, a 2+ case, and a 3+ case, respectively. In this additional non-limiting example experiment, format in invasive breast cancer tissues used for this study were absent of identifiers, except for the histological diagnosis. As depicted in FIGS. 30A, 30B, 30C, and 30D, four typical HER2 0-3+ breast cancer excision cases in our institution were selected. Formalin-fixed paraffin-embedded breast cancers were sectioned at 4 μm and 16 serial section slides were prepared for each case.

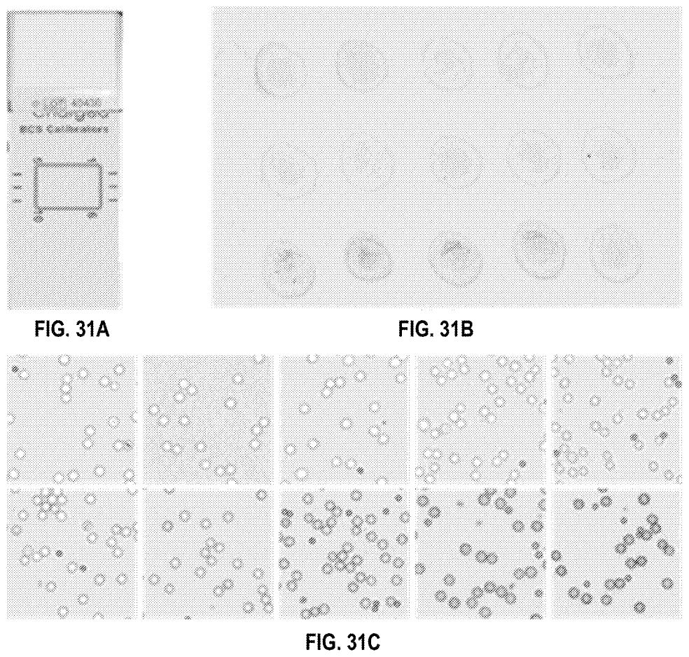

Referring to FIGS. 31A, 31B, and 31C, depicted is an example HER2 IHC stained IHC calibrator. FIG. 31A depicts an image of the calibrator slide, FIG. 31B depicts a WSI scanned at 0.23 μm/pixel resolution (40× equivalent) and displayed at 0.5× magnification. The top and bottom lines of FIG. 31B are stained in 10 intensity levels. The middle line of FIG. 31B includes unstained microbeads. FIG. 31C depicts a WSI displayed at 20× magnification, level 1 to 5 (top left to right), 6 to 10 (bottom left to right). This additional non-limiting example experiment utilized the IHC calibrator slide for HER2 shown in FIGS. 31A, 31B, and 31C. The IHC calibrator slide includes microbeads coated with 10 different levels of peptide concentration and stained with DAB by IHC staining.

In this additional non-limiting example experiment, five slides comprising HER2 0-3+ slides and the calibrator slide were utilized as a dataset. To obtain the slides that reflect the daily variation in staining, the datasets were stained at different dates continuously using PATHWAY anti-HER2/neu Antibody. In this additional non-limiting example experiment, sixteen IHC stained datasets were prepared, and the WSIs were obtained by a WSI scanner (e.g., a scanner device 125 of FIG. 1) at 0.23 μm/pixel resolution (40× equivalent). Exported bitmap image data were used for evaluation, and QuPath was utilized as an automated image analysis software for HER2 score classification (e.g., which may be executed by the data processing system 105).

Referring to FIG. 32 in the context of the components described in connection with FIG. 1, in this additional non-limiting example experiment, the process 3200, similar to the process 2000 of FIGS. 20A and 20A, and may be implemented by the data processing system 105 of FIG. 1, was utilized to perform standardization between slide images. As described herein, the process 3200 includes a preparation stage (e.g., similar to the preparation stage shown in FIG. 20A) and an evaluation stage (e.g., similar to the evaluation stage shown in FIG. 20B). In brief overview of the process 3200, a reference color intensity is determined in steps 3205 and 3210. A target color intensity of DAB is determined in step 3216, and used in the standardization procedure described herein. Step 3220 includes adjusting the thresholds (e.g., determined in step 3215) for classifying DAB membranous intensities using the parameters obtained from the calibrator slide. Step 3225 includes correcting the color intensity of the image using those parameters.

The process 3200 may be utilized in connection with the color un-mixing method described herein above. The color un-mixing method, which is based on the optical model, can be used to separate a color image of multi-stained specimens into the components of stain intensities representing the single stains, in our case, DAB and hematoxylin. The detected intensities of light transmitted through a specimen are described by Lambert-Beer's Law, known as the light transport formula through a homogenous absorbing medium. A color image is converted into the optical density space using the equations described above in connection with the non-limiting example experiment above, to determine a stain matrix. Determining the stain matrix can be useful, for example, because if the color of an image change depends on the staining batch, the same stain matrix cannot be used across images.

In step 3205, the data processing system can determine the stain matrices, e.g., V_{DAB} and V_{H}, as described herein, for the reference dataset using the tissue and calibrator images. To obtain V_{H}, the tissue slide scored as 0 is used. This slide can be stained with hematoxylin only, and V_{H} can be determined by calculating the unit vector from the average optical density of the tissue image. The matrix V_{DAB} can be determined by the data processing system 105 from the calibrator image. First, microbeads are detected by the data processing system 105 from the calibrator image using a Hough transform. The mean value of the central area of microbeads can be used for analysis. The calibrator slide can be stained with DAB, but the microbeads with lower stain intensity have a greyish tint in WSI. This is assumed to be due to the influence of light scattering by microbeads, and the optical density of calibrator images, O_{calibrator}, which is expressed by the model below:

$${O}_{calibrator} = {V}_{DAB} {C}_{DAB} + {V}_{scatter} {C}_{scatter}$$

In the above equation, the color vector of scattering, V_{scatter}, can be considered to be a wavelength-independent fixed value to represent a greyish tint. The matrix V_{DAB} can be calculated by semi-blindly separate V_{scatter} from O_{calibrator} using non-negative matrix factorization (NMF).

In step 3210, the matrices V_{H,R} and V_{DAB,R} can be calculated using the aforementioned for the reference dataset 3202 (which may be determined similarly to the reference dataset 2002 of the process 2000 in FIG. 20A, as described herein) in step 3205. Additionally, the matrix C_{DAB,R}, which is the DAB intensity of the calibrator image for reference, is estimated by color un-mixing, as described herein above. Similar techniques are utilized in step 3216 of the evaluation stage, to calculate V_{H,T}, V_{DAB,T}, and C_{DAB,T} for the target dataset 3212 (e.g., which may be obtained similarly to the target dataset 2012 of the process 2000 described in connection with FIG. 20B). A negative control can be used by the data processing system 105 to obtain V_{H,T}.

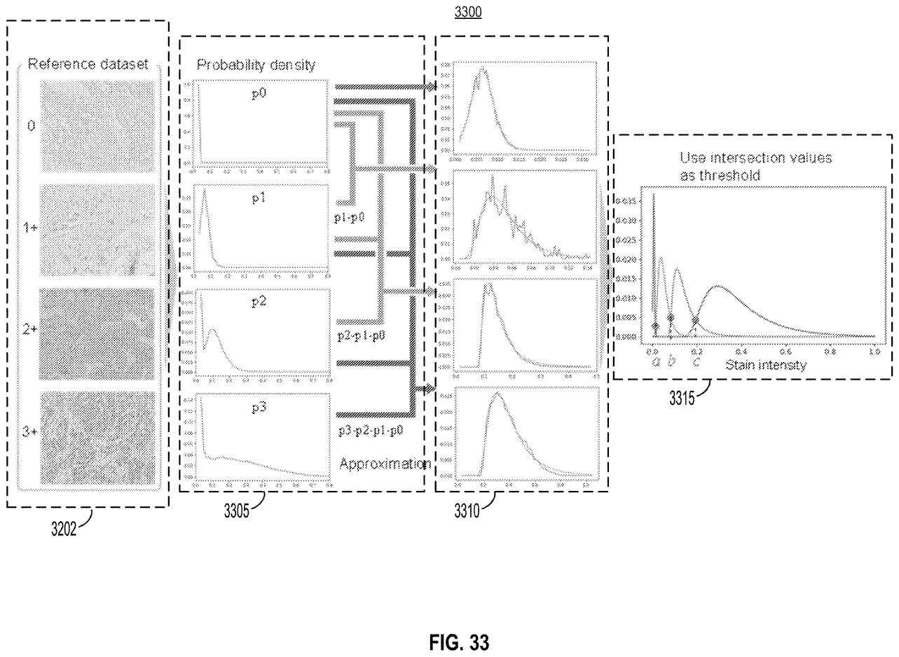

At step 3215, the data processing system can determine the thresholds of stain intensities from tissue images, as described in further detail in connection with FIG. 33. Referring to FIG. 33 in the context of the components of the system 100 of FIG. 1, illustrated is a data flow diagram of a process 3300 for calculating of the thresholds of stain intensities from tissue images. The process 3300 may be performed by the data processing system 105. As shown in the output graph 3315, the reference thresholds are defined as a, b, and c, which divide immunoscore 0, 1, 2, and 3 of the reference dataset 3202.

In the process 3300, color un-mixing is first performed to separate DAB and hematoxylin using the stain matrix obtained in step 3205 of FIG. 32. In step 3305, probability densities p0, p1, p2, and p3 are created from the DAB intensity of the membrane regions, as shown. In some implementations, the thresholds can be determined using intersection values of each probability density. For example, the intersection value of p0 and p1 can be determined as threshold a. The values for p0 and p1 can be close to a normal distribution, and the values of p2 and p3 can have a wide range and overlap, so the intersection value may be difficult to determine. In some implementations, additional distributions can be generated that may contain the intensity of one immunoscore by taking the difference in the probability densities. The distribution of score 0 is represented by p0 since it does not include the intensity of other scores. To represent the immunoscore 1, the value of p0 can be subtracted from p1. Likewise, the value of p0 and p1 can be subtracted from p2 to represent the immunoscore 2. To represent the immunoscore 3, subtract the values of p0, p1, and p2 can be subtracted from p3. Each of the distributions can be approximated using the Johnson SU distribution, and the intersection values of the distributions can be set as the thresholds a, b, and c.

Referring back to FIG. 32, at step 3217, the data processing system 105 can select a method for standardizing the images. As described herein, two implementations of the standardization procedure are provided for automated assessment. Two implementations may be selected from by the data processing system 105 depending on the capabilities of the automated image analysis software. For example, if the image analysis software is open-source or threshold adjustable, step 3220 of the process 3200 can be performed. Otherwise, the step 3225 of the process 3200 can be performed. In addition, color-corrected images obtained from step 3225 can be useful for confirmation performed by pathologists.

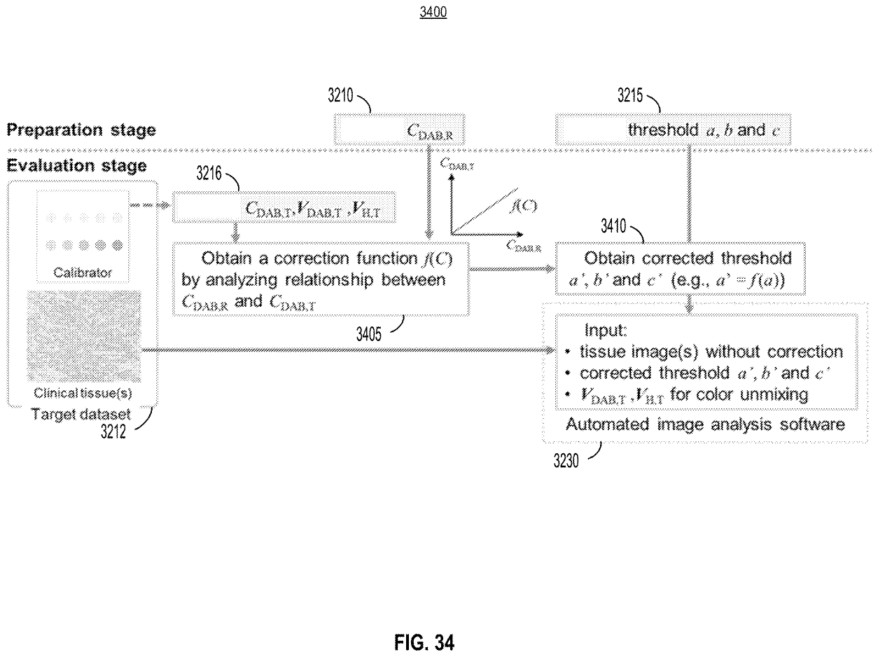

At step 3220 of the process 3200, the data processing system can correct the reference thresholds determined in step 3215 to the original tissue image for testing. To do so, the data processing system 105 can perform the steps of the process 3400 described in connection with FIG. 34. Referring to FIG. 34 in the context of the components described in connection with FIG. 1, depicted is a data flow diagram 3400 of a first implementation of the process 3200 described in connection with FIG. 32 including correcting reference thresholds to adapt to original tissue images. The steps of the data flow diagram 3400 can be performed when step 3220 is selected in step 3217 of the process 3200.

In step 3405, a correction function for intensity is calculated by the data processing system by analyzing the relationship between C_{DAB,R} and C_{DAB,T}. To remove outliers, the data of medium intensity can be used in a regression analysis from the original intensity data. For this purpose, both C_{DAB,R} and C_{DAB,T} can be sorted in intensity order. The median intensity and n intensities of both larger and smaller from the median intensity are extracted from C_{DAB,R} and C_{DAB,T}. As a result, two (2n+1)×Nm data C′_{DAB,R} and C′_{DAB,T} are generated. Here, Nm describes the number of detected microbeads. In this study, n was empirically set to 50. Linear function ƒ(C) is obtained by the regression analysis, by minimizing

$${\vert {C}_{DAB , T}^{′} - f ( {C}_{DAB , R}^{′} ) \vert}^{2} .$$

At step 3410, the data processing system 105 can use the obtained correction function ƒ(C) to map the stain intensity of the reference dataset (e.g., the reference dataset 3202) to that of the target dataset 3212. The data processing system 105 can then calculate corrected thresholds for the target dataset a′, b′, and c′ by mapping the reference thresholds through the correction function (e.g., a′=ƒ(a), etc.). In this implementation of step 3230, a score classification can be performed using the automated image analysis software, the corrected thresholds, and the tissue images are provided as input without correction. The stain matrix can also be provided for the color un-mixing techniques implemented in the automated image analysis software, as described herein.

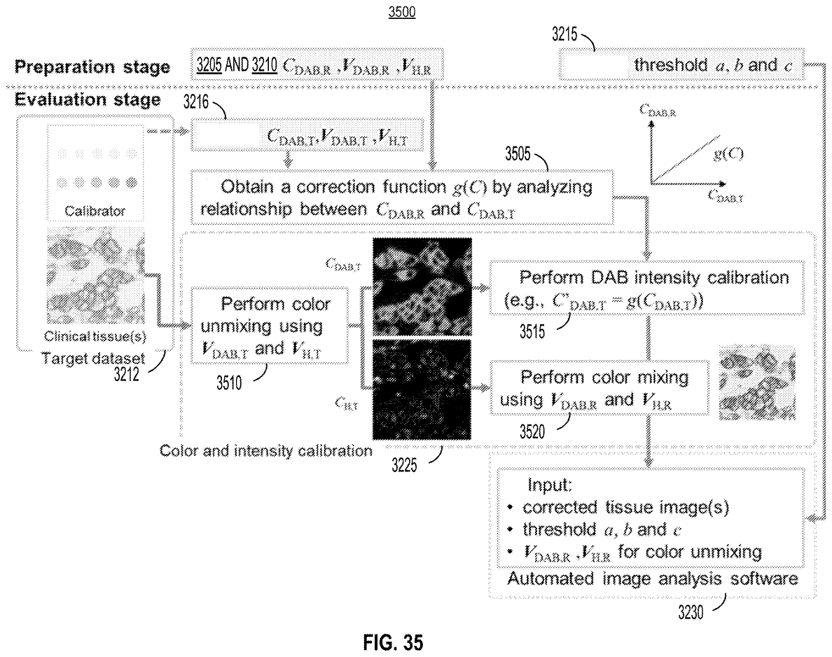

Referring back to FIG. 32 in the context of the system 100 described in connection with FIG. 1, the data processing system 105 can perform step 3225 if the corresponding method has been selected at step 3217. Step 3225 can include performing the process shown in the flow diagram 3500 of FIG. 35. Referring to FIG. 35, depicted is a data flow diagram 3500 of a second implementation of the process 3200 described in connection with FIG. 32 including correcting the color and intensity of tissue images. This example implementation may be performed by the data processing system 105, as described herein.

As described above, the process shown in the data flow diagram 3500 can be used to correct the color and intensity of tissue images. To do so, the data processing system 105 can calculate a correction function for intensity with the calibrator in the same manner as described in connection with FIG. 34, above. At step 3505, the data processing system 105 can calculate the linear function g(C) by minimizing |C_{DAB,R}−g(C_{DAB,T})|². The data processing system 105 can utilize the correction function to map the stain intensity of the target dataset 3212 to that of the reference dataset (e.g., the reference dataset 3202 of FIG. 32).

Since the calibrator and the tissue were stained and scanned together, the same V_{DAB} obtained from the calibrator image mentioned in the previous section can be utilized. Color correction of the tissue image is performed in three steps. First, at step 3510, the data processing system 105 can perform color un-mixing of the tissue image to separate C_{DAB,T} and C_{H,T} using V_{DAB,T} and V_{H,T} detected from the target dataset. Next, step 3515, the data processing system 105 can perform stain intensity calibration. Since the DAB intensity is targeted for HER2 score classification, the DAB intensity can be corrected without changing hematoxylin intensity. The data processing system 105 can correct C_{DAB,T} to C′_{DAB,T} by applying the correction function g(C). Finally, at step 3520, the data processing system 105 can perform color mixing to obtain the corrected color image using the following equation.

$${I}_{corrected} = {I}_{0} \cdot {10}^{- ( {V}_{DAB , R} {C}_{DAB , T}^{′} + {V}_{H , R} {C}_{H , T} )}$$

In the above equation, I_{corrected} is the pixel value of the corrected image. Here, the matrices V_{DAB,R} and V_{H,R} obtained from the reference dataset can be used by the data processing system 105 to correct the intensity and the color of tissue images. At step 3230, the tissue images after correction are provided to the automated image analysis software, which uses the reference thresholds a, b, and c. The stain matrix can also be provided for the color un-mixing techniques implemented in the automated image analysis software, as described herein.

At step 3230 of the process 3200, the data processing system 105 can execute automated image analysis software, which may be utilized in connection with the image standardization techniques described herein. The automated image analysis software can execute a cell detection algorithm using the input image as input, and can divide the cell regions into the nucleus, cytoplasm, and membrane. The data processing system 105 (or one or more components thereof) can execute a color un-mixing algorithm to separate signals of DAB from hematoxylin. Using these techniques, the data processing system 105 can derive the stain intensities in the target region of the images. To obtain HER2 status, the data processing system 105 can perform intensity classification according to user-set intensity thresholds. The DAB intensity of the membrane can be classified to immunoscore (0, 1, 2, or 3 corresponding to no, weak, moderate, or strong stain) according to intensity thresholds. Finally, the data processing system 105 can determine the HER2 status by the percentage of tumor cells according to the techniques described herein.

Referring back to FIG. 1 in the context of the additional non-limiting example experiment described herein above, provided are example experimental results of the additional non-limiting example experiment generated using the techniques described herein. To generate the results, one of the 16 datasets described herein above was regarded as a reference dataset, and the remaining 15 datasets were regarded as target datasets for HER2 test. On the reference dataset, regions of interest used for assessment and determining reference thresholds were selected. Reference thresholds were fixed and used commonly regardless of the datasets to compare with the proposed methods. Table 7 below shows the combination of the input images and thresholds for three methods.

TABLE 7
Combination of input images and threshold values

| | Alternative | Method of | Method of |
|---|---|---|---|
| | approach | step 3220 | step 3225 |
| Input images | Original image | Original image | Corrected image |
| Thresholds | Fixed values | Corrected values | Fixed values |
| | obtained from | (Different values | obtained from |
| | reference dataset | for each dataset) | reference dataset |

To calculate the results of the additional non-limiting example experiment, QuPath was used as an automated image analysis software for HER2 score classification. The software executed by the data processing system 105 may be semi-automatic, and can enable setting color vectors for color un-mixing and thresholds for classification. DAB membrane staining is classified as immunoscore (0, 1, 2, or 3) according to the thresholds. The data processing system 105 can calculate the histoscore (H-score), which is one of the evaluation indexes in HER2 assessment, by solving the following equation.

$$H - score = P \times I$$

In the above equation, I is immunoscore (0, 1, 2, or 3) classified according to the thresholds and P is the percentage of immunoreactive tumor cells, respectively. The example results of the additional non-limiting example experiment are as follows. Table 8 shows the result of the HER2 score classification. Using an alternative approach that does not implement the present techniques, there were some misclassified slides (e.g., 7/60 slides). Using the techniques described herein, the misclassification was greatly eliminated (0/60 slide when implementing the approach described in step 3220 of FIG. 32, 1/60 slide when implementing the approach described in step 3225 of FIG. 32). The SD of the H-score became smaller in both implementations (e.g., step 3220 and step 3225).

Table 9 shows the results of an analysis of variance and Table 10 shows the result of Tukey HSD test. An analysis of variance showed that the effect of the method was significant in 1+ case, F (2, 40)=10.75, P=0.000, in 2+ case, F (2, 40)=11.79, P=0.000 and in 3+ case, F (2,40)=5.14,P=0.010. Post hoc analyses using the Tukey's HSD test for significance indicated showed that the method was significant in 2+ between proposed method 1 and previous method (P=0.001, 95% C.I.=[6.34, 24.36]), proposed method 2 and previous method (P=0.001, 95% C.I.=[6.83, 24.85]), in 3+ between proposed method 1 and previous method (P=0.015, 95% C.I.=[1.70, 18.39]), proposed method 2 and previous method (P=0.033, 95% C.I.=[0.60, 17.28]).

TABLE 8
Results of score classification

| | | Alternative method | | Method of step 3220 | | Method of step 3225 | |
|---|---|---|---|---|---|---|---|
| HER2 | | Concordance | H-score | Concordance | H-score | Concordance | H-score |
| status | Num. | (Rate (%)) | SD | (Rate (%)) | SD | (Rate (%)) | SD |
| 0 | 15 | 15 (100.0) | 2.5 | 15 (100.0) | 0.1 | 15 (100.0) | 0.1 |
| 1+ | 15 | 12 (80.0) | 5.3 | 15 (100.0) | 6.4 | 15 (100.0) | 3.7 |
| 2+ | 15 | 11 (73.3) | 10.8 | 15 (100.0) | 9.3 | 14 (93.3) | 9.2 |
| 3+ | 15 | 15 (100.0) | 10.4 | 15 (100.0) | 9.1 | 15 (100.0) | 7.5 |

TABLE 9
Results of an analysis of variance to see if there
was a significant difference between three methods;

| HER2 | | |
|---|---|---|
| status | F | P |
| 0 | 2.31 | .112 |
| 1+ | 10.75 *** | .000 |
| 2+ | 11.79 *** | .000 |
| 3+ | 5.14 * | .010 |

* P < 0.05
*** P < 0.001.

TABLE 10
Results of post hoc analysis in cases with significant difference;

| | | | | | 95% confidence | |
|---|---|---|---|---|---|---|
| HER2 | | | | | interval | |
| status | group 1 | group 2 | Mean diff. | P | Lower | Upper |
| 1+ | Step 3220 | Step 3225 | 6.57 ** | .006 | 1.73 | 11.41 |
| | Step 3220 | Alternative | 8.91 ** | .001 | 4.07 | 13.75 |
| | Step 3225 | Alternative | 2.34 | .476 | −2.50 | 7.18 |
| 2+ | Step 3220 | Step 3225 | −0.49 | .900 | −9.50 | 8.52 |
| | Step 3220 | Alternative | 15.35 ** | .001 | 6.34 | 24.36 |
| | Step 3225 | Alternative | 15.84 ** | .001 | 6.83 | 24.85 |
| 3+ | Step 3220 | Step 3225 | 1.10 | .900 | −7.24 | 9.45 |
| | Step 3220 | Alternative | 10.04 * | .015 | 1.70 | 18.39 |
| | Step 3225 | Alternative | 8.94 * | .033 | 0.60 | 17.28 |

* P < 0.05
** P < 0.01

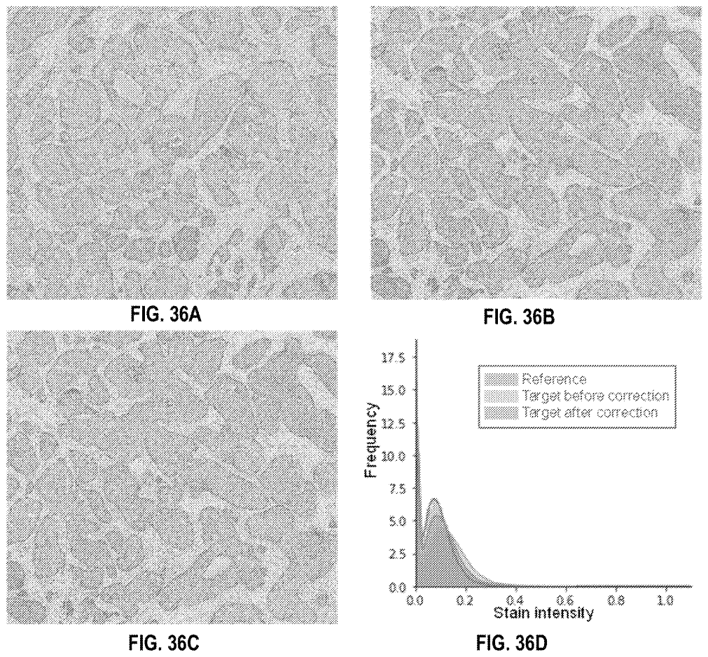

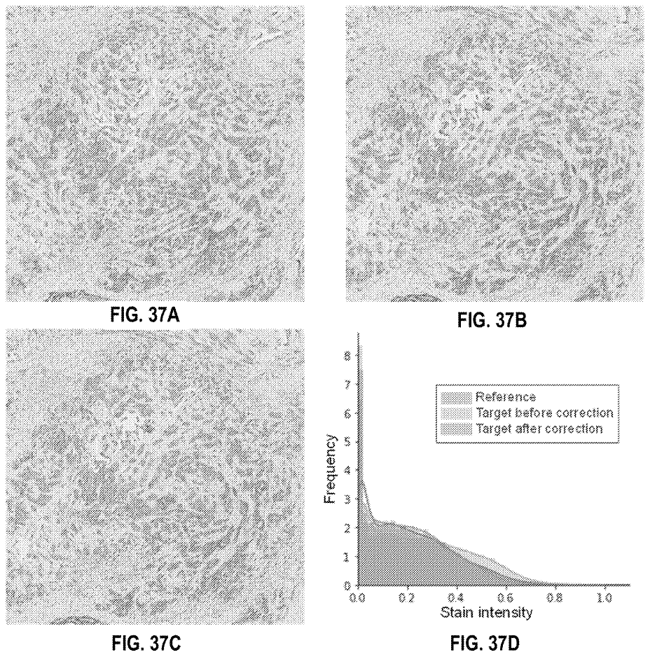

FIGS. 36A, 36B, 36C, and 36D, and FIGS. 37A, 37B, 37C, and 37D show examples of color correction resulting from performing the implementation of the process 3220 in which step 3225 is selected. FIGS. 36A, 36B, 36C, and 36D depict examples of color correction in 2+ cases. FIG. 36A depicts a reference image, FIG. 36B depicts a target image before correction, FIG. 36C depicts the target image after correction, and FIG. 36D depicts plots of histograms of DAB membranous stain comparing the reference and target images. FIGS. 37A, 37B, 37C, and 37D depict examples of color correction in 3+ cases. FIG. 37A depicts a reference image, FIG. 37B depicts a target image before correction, FIG. 37C depicts the target image after correction, and FIG. 37D depicts plots of histograms of DAB membranous stain comparing the reference and target images.

The color of the target images were corrected using the techniques described herein to approach the reference images, and the histograms were also changed to fit the reference image. Table 11 below shows a comparison of the similarity of the histograms. For the correlation coefficient, a value closer to 1 indicates a higher similarity. For Chi-square analysis, a lesser value indicates a higher value for the similarity in results. Based on the correlation coefficient, the similarity between the reference and the correction target image was high even before correction. In this additional non-limiting example experiment, since all datasets were stained at one institution, the variation in staining was small. The Chi-square result showed that the value after correction was smaller than that before correction in 1+ and 2+ cases. The T-test showed that the effect of the method was significant in 2+ cases, t=−4.90, P=0.000 with the correlation coefficient, t=3.58, P=0.002 with the Chi-square.

TABLE 11
Comparison of histogram similarity using
correlation coefficient and Chi-square;

| | Similarity | | | | |
|---|---|---|---|---|---|
| HER2 | | Reference vs | Reference vs | T-test | |
| status | Method | Before | After | t | P |
| 1+ | correlation | 0.98 ± 0.02 | 0.99 ± 0.02 | −0.30 | .766 |
| 2+ | coefficient | 0.95 ± 0.03 | 0.98 ± 0.02 | −4.90 *** | .000 |
| 3+ | | 0.98 ± 0.01 | 0.98 ± 0.02 | 1.16 | .265 |
| 1+ | Chi-square | 0.14 ± 0.19 | 0.05 ± 0.06 | 1.52 | .151 |
| 2+ | | 0.43 ± 0.35 | 0.08 ± 0.09 | 3.85 ** | .002 |
| 3+ | | 0.04 ± 0.04 | 0.05 ± 0.04 | −0.63 | .542 |

** P < 0.01
*** P < 0.001.

The techniques described herein utilizing the calibrator slide allows standardization of color and intensity of WSI for HER2 test, which vary depending on staining process. In the context of this additional non-limiting example experiment, the techniques described herein show that misclassification was almost eliminated, and that the SD of H-score became smaller for most cases by applying the present techniques, reducing variation in score classification. A significant difference was observed between alternative approaches and the present techniques, in 2+ and 3+ cases. In addition, the present techniques can be used to obtain thresholds from tissue images. The thresholds provided the correct HER2 status in combination with our standardization methods.

Minimal modifications are required to integrate the techniques described herein into clinical workflows, including conducting the same staining protocol to the calibrator slide together with target tissue slides. By changing the antibody of the calibrator slide, the techniques described herein can be extended to the HER2 test in gastric cancer and other IHC tests in breast cancer, such as Ki67 and PD-L1. One of the advantages of the calibrator described herein is that there are 10 intensity levels. Compared to the usual negative and positive controls representing typical 0 and 3+ HER2 cases, the difference in intensity from 0 to 3+ can be observed in detail. Regarding the acquisition of the correction function ƒ(C) and g(C), it is possible to find the correspondence between C_{DAB,R} and C_{DAB,T} in detail thanks to a large number of levels on the calibrator.

While the disclosure has been described with respect to specific embodiments, one skilled in the art will recognize that numerous modifications are possible. Embodiments of the disclosure can be realized using a variety of computer systems and communication technologies including but not limited to specific examples described herein. Embodiments of the present disclosure can be realized using any combination of dedicated components and/or programmable processors and/or other programmable devices. The various processes described herein can be implemented on the same processor or different processors in any combination. Where components are described as being configured to perform certain operations, such configuration can be accomplished, e.g., by designing electronic circuits to perform the operation, by programming programmable electronic circuits (such as microprocessors) to perform the operation, or any combination thereof. Further, while the embodiments described above may make reference to specific hardware and software components, those skilled in the art will appreciate that different combinations of hardware and/or software components may also be used and that particular operations described as being implemented in hardware might also be implemented in software or vice versa.

Computer programs incorporating various features of the present disclosure may be encoded and stored on various computer readable storage media; suitable media include magnetic disk or tape, optical storage media such as compact disk (CD) or DVD (digital versatile disk), flash memory, and other non-transitory media. Computer readable media encoded with the program code may be packaged with a compatible electronic device, or the program code may be provided separately from electronic devices (e.g., via Internet download or as a separately packaged computer-readable storage medium).

Thus, although the disclosure has been described with respect to specific embodiments, it will be appreciated that the disclosure is intended to cover all modifications and equivalents within the scope of the following claims.

Aspects can be combined and it will be readily appreciated that features described in the context of one aspect can be combined with other aspects. Aspects can be implemented in any convenient form. For example, by appropriate computer programs, which may be carried on appropriate carrier media (computer readable media), which may be tangible carrier media (e.g. disks) or intangible carrier media (e.g. communications signals). Aspects may also be implemented using a suitable apparatus, which can take the form of one or more programmable computers running computer programs arranged to implement the aspect. As used in the specification and in the claims, the singular form of ‘a’, ‘an’, and ‘the’ include plural referents unless the context clearly dictates otherwise.

What is claimed is:

1. A method for performing color calibration using calibration slides to automate detection, the method comprising:
receiving, by one or more processors coupled to a non-transitory memory, a plurality of calibration images from a scanner, each of the plurality of calibration images depicting a portion of a respective stained tissue sample having a known score value;
determining, by the one or more processors, a correction function for the scanner based on the plurality of calibration images;
receiving, by the one or more processors, an unscored slide image depicting a portion of stained tissue, the unscored slide image captured by the scanner; and
presenting, by the one or more processors in a graphical user interface of an application executing on the one or more processors, a score for the unscored slide image generated using the correction function for the scanner.

2. The method of claim 1, further comprising determining, by the one or more processors, the score for the unscored slide image based on the correction function.

3. The method of claim 1, wherein each of the plurality of calibration images are selected to correspond to a respective score value.

4. The method of claim 1, wherein determining the correction function comprises performing a color calibration process.

5. The method of claim 4, wherein the color calibration process comprises calculating, by the one or more processors, a color correction matrix that is applied to the unscored slide image.

6. The method of claim 1, wherein determining the correction function comprises determining, by the one or more processors, one or more color thresholds corresponding to the plurality of calibration images.

7. The method of claim 1, further comprising annotating, by the one or more processors, the unscored slide image based on the correction function.

8. The method of claim 1, wherein determining the correction function comprises determining one or more intensity values over a multiple day period.

9. The method of claim 1, further comprising:
receiving, by the one or more processors, a second unscored slide image generated by a second scanner during a second time period;
identifying, by the one or more processors, a second correction function associated with the second scanner and the second time period; and
presenting, by the one or more processors, a second score for the second unscored slide image in the application generated using the second correction function.

10. The method of claim 1, wherein the plurality of calibration images each correspond to a respective HER2 score.

11. A system for performing color calibration using control tissue to automate detection, the system comprising:
one or more processors coupled to a non-transitory memory, the one or more processors configured to:
receive a plurality of calibration images from a scanner, each of the plurality of calibration images depicting a portion of a respective stained tissue sample having a known score value;
determine a correction function for the scanner based on the plurality of calibration images;
receive an unscored slide image depicting a portion of stained tissue, the unscored slide image captured by the scanner; and
present, in a graphical user interface of an application executing on the one or more processors, a score for the unscored slide image generated using the correction function for the scanner.

12. The system of claim 11, wherein the one or more processors are further configured to determine the score for the unscored slide image based on the correction function.

13. The system of claim 11, wherein each of the plurality of calibration images are selected to correspond to a respective score value.

14. The system of claim 11, wherein to determine the correction function, the one or more processors are further configured to perform a color calibration process.

15. The system of claim 14, wherein the one or more processors are further configured to calculate a color correction matrix that is applied to the unscored slide image.

16. The system of claim 11, wherein to determine the correction function, the one or more processors are further configured to determine one or more color thresholds corresponding to the plurality of calibration images.

17. The system of claim 11, wherein the one or more processors are further configured to annotate the unscored slide image based on the correction function.

18. The system of claim 11, wherein to determine the correction function, the one or more processors are further configured to determine one or more intensity values over a multiple day period.

19. The system of claim 11, wherein the one or more processors are further configured to:
receive a second unscored slide image generated by a second scanner during a second time period;
identify a second correction function associated with the second scanner and the second time period; and
present a second score for the second unscored slide image in the application generated using the second correction function.

20. The system of claim 11, wherein the plurality of calibration images each correspond to a respective HER2 score.
